(19) United States
(12) Patent Application Publication
(10) Pub. No.: US 2022/0133742 A1
(43) Pub. Date: May 5, 2022

(54) METHODS OF TREATING CONGENITAL ADRENAL HYPERPLASIA

(71) Applicant: Spruce Biosciences, Inc., Daly City, CA (US)

(72) Inventors: Sangita GHOSH, San Francisco, CA (US); Christopher BARNES, San Francisco, CA (US); Michael HUANG, San Francisco, CA (US)

(21) Appl. No.: 17/578,149

(22) Filed: Jan. 18, 2022

Related U.S. Application Data
(63) Continuation of application No. PCT/US2020/042820, filed on Jul. 20, 2020.
(60) Provisional application No. 62/876,176, filed on Jul. 19, 2019.
(60) Provisional application No. 63/047,822, filed on Jul. 2, 2020.

(51) Int. Cl. A61K 31/573; A61K 31/5377; A61P 5/38; A61K 45/06

(52) U.S. Cl. CPC A61K 31/573; A61K 45/06; A61P 5/38; A61K 31/5377

(57) ABSTRACT

Patients with congenital adrenal hyperplasia (CAH) need adequate care and treatment in order to lead normal lives. Hence, there is a need for new methods of treating CAH. This disclosure provides new compounds, salts, compositions and uses thereof in the treatment of CAH.

### CROSS-REFERENCE

This application is the continuation of International Application No. PCT/US2020/042820, filed on Jul. 20, 2020, which claims priority to U.S. Provisional Patent Application No. 62/876,176, filed Jul. 19, 2019, and U.S. Provisional Patent Application No. 63/047,822, filed on Jul. 2, 2020, each of which is entirely incorporated herein by reference.

### BACKGROUND

Congenital adrenal hyperplasia (CAH) is a group of rare inherited autosomal recessive disorders characterized by a deficiency of one of the enzymes needed to make specific hormones. CAH affects the adrenal glands located at the top of each kidney. Normally, the adrenal glands are responsible for producing three different hormones: 1) glucocorticoids, which gauge the body's response to stress, illness, or injury; 2) mineralocorticoids, which regulate salt and water levels; and 3) androgens, which are male sex hormones. An enzyme deficiency may make the body unable to produce one or more of these hormones, which in turn may result in the overproduction of another type of hormone precursor in order to compensate for the loss.

### SUMMARY

Congenital Adrenal Hyperplasia (CAH) patients need adequate care and treatment in order to lead normal lives. Hence, there is a need for new methods of treating CAH. This disclosure provides new compounds, salts, compositions and uses thereof in the treatment of CAH.

In an aspect, the present disclosure provides methods for treating Congenital Adrenal Hyperplasia (CAH). The method comprises administering a steroid or a pharmaceutically acceptable salt thereof and a corticotropin-releasing factor type-1 (CRF₁) antagonist or a pharmaceutically acceptable salt thereof to a subject, wherein when said subject has a level of at least one steroid hormone that is less than two times an upper limit of a reference range of said at least one steroid hormone level, a dose of said steroid or a pharmaceutically acceptable salt thereof is reduced compared to a dose of said steroid or a pharmaceutically acceptable salt thereof administered to a CAH patient that does not receive said CRF₁ antagonist or a pharmaceutically acceptable salt thereof.

In some embodiments, the subject has a level of a second and different steroid hormone that is less than two times an upper limit of a reference range of said second and different steroid hormone level. In some embodiments, the steroid hormone is selected from the group consisting of adrenocorticotropic hormone (ACTH), androstenedione (A4), 17-hydroxyprogesterone (17-OHP), deoxycorticosterone, 11-deoxycortisol, cortisol, corticosterone, aldosterone, pregnenolone, 17α-hydroxy pregnenolone, progesterone, dehydroepiandrosterone, androstenediol, testosterone, dihydrotestosterone, estrone, estradiol, and estriol.

In some embodiments, the subject has a level of 17-OHP that is less than two times an upper limit of a reference range of 17-OHP and a level of ACTH that is less than two times an upper limit of a reference range of ACTH. In some embodiments, the subject has a level of 17-OHP that is less than two times an upper limit of a reference range of 17-OHP and a level of A4 that is less than two times an upper limit of a reference range of A4. In some embodiments, the subject has a level of A4 that is less than two times an upper limit of a reference range of A4 and a level of ACTH that is less than two times an upper limit of a reference range of ACTH. In some embodiments, the subject has a level of 17-OHP that is less than 1.75 times an upper limit of a reference range of 17-OHP and a level of ACTH that is less than 1.75 times an upper limit of a reference range of ACTH. In some embodiments, the subject has a level of 17-OHP that is less than 1.75 times an upper limit of a reference range of 17-OHP and a level of A4 that is less than 1.75 times an upper limit of a reference range of A4. In some embodiments, the subject has a level of A4 that is less than 1.75 times an upper limit of a reference range of A4 and a level of ACTH that is less than 1.75 times an upper limit of a reference range of ACTH. In some embodiments, the subject has a level of 17-OHP that is less than 1.5 times an upper limit of a reference range of 17-OHP and a level of ACTH that is less than 1.5 times an upper limit of a reference range of ACTH. In some embodiments, the subject has a level of 17-OHP that is less than 1.5 times an upper limit of a reference range of 17-OHP and a level of A4 that is less than 1.5 times an upper limit of a reference range of A4. In some embodiments, the subject has a level of A4 that is less than 1.5 times an upper limit of a reference range of A4 and a level of ACTH that is less than 1.5 times an upper limit of a reference range of ACTH. In some embodiments, the subject has a level of 17-OHP that is less than 1.25 times an upper limit of a reference range of 17-OHP and a level of ACTH that is less than 1.25 times an upper limit of a reference range of ACTH. In some embodiments, the subject has a level of 17-OHP that is less than 1.25 times an upper limit of a reference range of 17-OHP and a level of A4 that is less than 1.25 times an upper limit of a reference range of A4. In some embodiments, the subject has a level of A4 that is less than 1.25 times an upper limit of a reference range of A4 and a level of ACTH that is less than 1.25 times an upper limit of a reference range of ACTH. In some embodiments, the subject has a level of 17-OHP that is less than one times an upper limit of a reference range of 17-OHP and a level of ACTH that is less than one times an upper limit of a reference range of ACTH. In some embodiments, the subject has a level of 17-OHP that is less than one times an upper limit of a reference range of 17-OHP and a level of A4 that is less than one times an upper limit of a reference range of A4. In some embodiments, the subject has a level of A4 that is less than one times an upper limit of a reference range of A4 and a level of ACTH that is less than one times an upper limit of a reference range of ACTH.

In some embodiments, the methods comprise reducing said dose of said steroid or a pharmaceutically acceptable salt thereof in a 5 mg increment.

In another aspect, the present disclosure provides methods for treating Congenital Adrenal Hyperplasia (CAH). The methods comprise administering a steroid or a pharmaceutically acceptable salt thereof and a corticotropin-releasing factor type-1 (CRF1) antagonist or a pharmaceutically acceptable salt thereof to a subject, wherein when said subject has a level of at least one steroid hormone that is more than an upper limit of a reference range of said at least one steroid hormone level, a dose of said steroid or a pharmaceutically acceptable salt thereof is not reduced compared to a dose of said steroid or a pharmaceutically acceptable salt thereof administered to a CAH patient that does not receive said CRF1 antagonist or a pharmaceutically acceptable salt thereof.

In some embodiments, the subject has a level of said at least one steroid hormone that is more than two times an upper limit of a reference range of said at least one steroid hormone level. In some embodiments, the subject has a level of said at least one steroid hormone that is more than 1.75 times an upper limit of a reference range of said at least one steroid hormone level. In some embodiments, the subject has a level of said at least one steroid hormone that is more than 1.5 times an upper limit of a reference range of said at least one steroid hormone level. In some embodiments, the subject has a level of said at least one steroid hormone that is more than 1.25 times an upper limit of a reference range of said at least one steroid hormone level.

In some embodiments, the steroid hormone is selected from the group consisting of adrenocorticotropic hormone (ACTH), androstenedione (A4), 17-hydroxyprogesterone (17-OHP), deoxycorticosterone, 11-deoxycortisol, cortisol, corticosterone, aldosterone, pregnenolone, 17α-hydroxy pregnenolone, progesterone, dehydroepiandrosterone, androstenediol, testosterone, dihydrotestosterone, estrone, estradiol, and estriol.

In some embodiments, the methods further comprise maintaining said dose of said steroid or a pharmaceutically acceptable salt thereof until said levels of at least two steroid hormones are less than 2 times said upper limit of said reference ranges of said two steroid hormones respectively. In some embodiments, the methods further comprise reducing said dose of said steroid or a pharmaceutically acceptable salt thereof in a 5 mg increment when said levels of at least two steroid hormones are less than 2 times said upper limit of said reference ranges of said two steroid hormones respectively.

In some embodiments, the at least two steroid hormones comprise 17-OHP and ACTH. In some embodiments, the at least two steroid hormones comprise 17-OHP and A4. In some embodiments, the at least two steroid hormones comprise A4 and ACTH. In some embodiments, the reference range of ACTH or A4 level of said subject is controlled by a factor selected from the group consisting of age, sex, menopausal status, laboratory setting, time of a day and combinations thereof.

In some embodiments, the level of ACTH or A4 is 2 times, 1.5 times, 1.25 times, or 1 times said upper limit of said normal range of ACTH or A4 level of said subject. In some embodiments, the amount of said steroid or a pharmaceutically acceptable salt thereof comprises a range of about 10 mg to about 80 mg of hydrocortisone equivalents, wherein said steroid or a pharmaceutically acceptable salt thereof is administered daily. In some embodiments, the amount of said CRF₁ antagonist or a pharmaceutically acceptable salt thereof comprises a range of about 10 mg to about 200 mg, wherein said CRF₁ antagonist or a pharmaceutically acceptable salt thereof is administered daily.

In some embodiments, the subject is treated for a period of about 4 weeks to about 36 weeks.

In some embodiments, the subject is treated for a period of about 4 weeks to about 36 weeks, said subject is reassessed to determine if a different method of treating CAH should be administered.

In some embodiments, the CRF₁ antagonist or pharmaceutically acceptable salt thereof is selected from the group consisting of: Antalarmin hydrochloride, Pfizer CP 154526, CP 376395 hydrochloride, NBI 27914 hydrochloride, NBI 35965 hydrochloride, NGD 98-2 hydrochloride, Pexacerfont, R 121919 hydrochloride, SNO03.

In some embodiments, the CRF₁ antagonist is a compound of Formula (I):

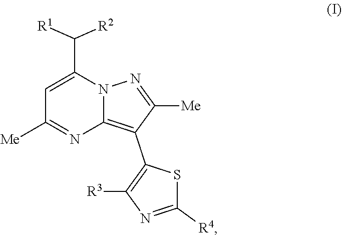

or a pharmaceutically acceptable salt thereof, wherein:
R¹ and R² are independently ethyl or n-propyl;
R³ is hydrogen, Cl, Br, methyl, trifluoromethyl, or methoxy; and
R⁴ is hydrogen, Br, R^{a}R^{b}N—, methoxymethyl, n-butyl, acetamido, pyridin-4-yl, morpholin-4-yl, and

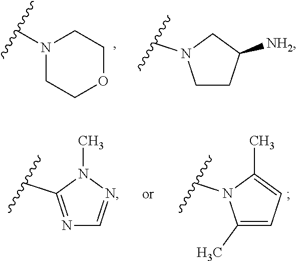

R^{a} and R^{b} are independently hydrogen, C₁-C₃alkyl, H₂NCH₂CH₂—, (CH₃)₃COC(O)NHCH₂CH₂—, or CH₃CH₂CH₂NHCH₂CH₂—.

In some embodiments, R³ is Cl, Br, methyl, or trifluoromethyl.

The method of claim 38 or 39, wherein R³ is Cl, Br, or methyl.

In some embodiments, R⁴ is Br, R^{a}R^{b}N—, pyridin-4-yl, morpholin-4-yl, or

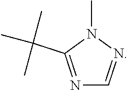

In some embodiments, R⁴ is morpholin-4-yl or

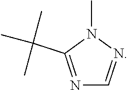

In some embodiments, R⁴ is hydrogen, Br, R^{a}R^{b}N— and R^{a} and R^{b} are independently C₁-C₃alkyl.

In some embodiments the compound is

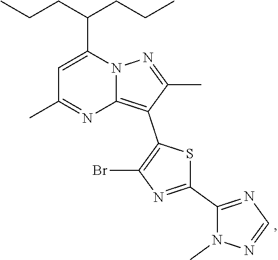 [SMILES: CCCC(CCC)c1cc(C)nc2c(-c3sc(-c4ncnn4C)nc3Br)c(C)nn12]

or a pharmaceutically acceptable salt thereof.

In some embodiments the compound is

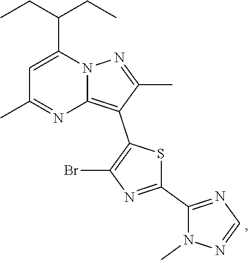 [SMILES: CCC(CC)c1cc(C)nc2c(-c3sc(-c4ncnn4C)nc3Br)c(C)nn12]

or a pharmaceutically acceptable salt thereof.

In some embodiments the compound is

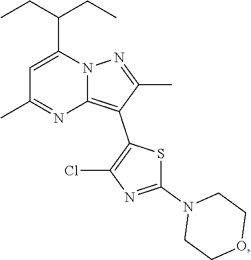 [SMILES: CCC(CC)c1cc(C)nc2c(-c3sc(N4CCOCC4)nc3Cl)c(C)nn12]

or a pharmaceutically acceptable salt thereof.

In some embodiments, the CRF1 antagonist or pharmaceutically acceptable salt is administered in a dose of about 50 mg to about 200 mg total daily dose to the subject.

In some embodiments, the CRF1 antagonist or pharmaceutically acceptable salt is administered in a dose of about 200 mg total daily dose to the subject. In some embodiments, wherein the CRF1 antagonist or pharmaceutically acceptable salt is administered in a dose of about 150 mg total daily dose to the subject.

In some embodiments, the CRF1 antagonist or pharmaceutically acceptable salt is administered in a dose of about 100 mg total daily dose to the subject. In some embodiments, the CRF1 antagonist or pharmaceutically acceptable salt is administered in a dose of about 50 mg total daily dose to the subject.

In some embodiments, the CRF1 antagonist or pharmaceutically acceptable salt is in the form of microparticles. In some embodiments, the average size of the microparticles is between about 1 μm to about 20 μm. In some embodiments, the average size of the microparticles is between about 1 μm to about 20 μm. In some embodiments, the average size of the microparticles is between about 5 μm to about 15 μm. In some embodiments, the average size of the microparticles is less than about 10 μm.

In some embodiments, the CRF1 antagonist or pharmaceutically acceptable salt thereof is administered as a pharmaceutical composition. In some embodiments, the steroid or a pharmaceutically acceptable salt thereof is administered as a pharmaceutical composition.

In some embodiments, the pharmaceutical composition is in the form of a capsule or a tablet. In some embodiments, the capsule is a hard gelatin capsule. In some embodiments, the capsule is a soft gelatin capsule. In some embodiments, the capsule is formed using materials selected from the group consisting of natural gelatin, synthetic gelatin, pectin, casein, collagen, protein, modified starch, polyvinylpyrrolidone, acrylic polymers, cellulose derivatives, and any combinations thereof.

In some embodiments, the pharmaceutical composition is free of additional excipients. In some embodiments, the pharmaceutical composition further comprises one or more pharmaceutically acceptable excipients. In some embodiments, the pharmaceutical composition is in the form of a tablet. In some embodiments, the pharmaceutical composition further comprises one or more pharmaceutically acceptable excipients. In some embodiments, the steroid or pharmaceutically acceptable salt thereof is a glucocorticoid or a pharmaceutically acceptable salt thereof. In some embodiments, the glucocorticoid or a pharmaceutically acceptable salt thereof is prednisone, cortisone, prednisolone, triamcinolone, methylprednisolone, betamethasone, dexamethasone, hydrocortisone or a pharmaceutically acceptable salt thereof. In some embodiments, the levels of steroid hormones in the subject are determined from a biological sample from the subject. In some embodiments, the biological sample is selected from the group of blood, blood fractions, plasma, serum, and saliva. In some embodiments, the biological sample is obtained non-invasively.

In some embodiments, the subject is tested in the morning. In some embodiments, subject is tested to determine morning levels of A4 and ACTH. In some embodiments, the subject is a pediatric patient. In some embodiments, the subject is from about newborn to about 18 years old.

In some embodiments, the subject is an adult patient. In some embodiments, the steroid and the CRF1 antagonist are administered concurrently. In some embodiments, the steroid and the CRF1 antagonist are administered in one pharmaceutical composition.

In some embodiments, the steroid and the CRF1 antagonist are administered concurrently in separate pharmaceutical compositions. In some embodiments, the steroid and the CRF1 antagonist are administered sequentially. In some embodiments, the steroid and the CRF1 antagonist are administered sequentially within 24 hours. In some embodiments, the steroid and the CRF1 antagonist are administered sequentially within 8 hours. In some embodiments, the steroid and the CRF1 antagonist are administered sequentially within 2 hours. In some embodiments, the steroid and the CRF1 antagonist are administered sequentially within 10 minutes.

In some embodiments, CAH is classic CAH. In some embodiments, CAH is non-classic CAH.

In another aspects, the present disclosure provides compositions comprising a steroid or a pharmaceutically acceptable salt thereof and a corticotropin-releasing factor type-1 (CRF₁) antagonist or a pharmaceutically acceptable salt thereof, wherein said CRF₁ antagonist or pharmaceutically acceptable salt thereof is selected from the group consisting of: Antalarmin hydrochloride, Pfizer CP 154526, CP 376395 hydrochloride, NBI 27914 hydrochloride, NBI 35965 hydrochloride, NGD 98-2 hydrochloride, Pexacerfont, R 121919 hydrochloride, SNO03. In some embodiments, the steroid or a pharmaceutically acceptable salt thereof is exogenous glucocorticoids (GC) or a pharmaceutically acceptable salt thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.”), of which:

FIG. 1 shows the baseline upper limit of a normal range (ULN)/Target Multiples for Serum 17-OHP, ACTH and A4 by subgroup;

FIGS. 2A and 2B illustrate a time matched change of ACTH hormone profile;

FIGS. 3A and 3B illustrate a time matched change of 17-OHP hormone profile; and

FIGS. 4A and 4B illustrate a time matched change of ACTH hormone profile.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Congenital Adrenal Hyperplasia

Congenital adrenal hyperplasia (CAH) is a group of rare inherited autosomal recessive disorders characterized by a deficiency of one of the enzymes needed to make specific hormones. CAH effects the adrenal glands located at the top of each kidney. Normally, the adrenal glands are responsible for producing three different hormones: 1) glucocorticoids, which gauge the body's response to stress, illness or injury; 2) mineralocorticoids, which regulate salt and water levels; and 3) androgens, which are male sex hormones. An enzyme deficiency will make the body unable to produce one or more of these hormones, which in turn will result in the overproduction of another type of hormone precursor in order to compensate for the loss.

The most common cause of CAH is the absence of the enzyme 21-hydroxylase. Different mutations in the gene responsible for 21-hydroxylase result in different levels of the enzyme, producing a spectrum of effects. CAH due to 21-hydroxylase deficiency is responsible for 95% of all cases of CAH and is broken down further into two subcategories: classic CAH, which can be sub-divided into the salt-losing form or the simple-virilizing form, and non-classic CAH. Classic CAH is by far the more severe form and can result in adrenal crisis and death if not detected and treated. Non-classic CAH is milder and may or may not present symptoms. Since the absence of 21-hydroxylase makes these individuals unable to make the hormone cortisol, a glucocorticoid, and, in the case of salt-losing CAH, aldosterone (a mineralocorticoid), the excess steroid precursors, namely 17-hydroxyprogesterone (17-OHP), build up and are shunted down the androgen pathway resulting in excess androgen production which cause a variety of symptoms such as abnormal genital development in infant girls. There are other much rarer forms of CAH as well, including 11-β hydroxylase deficiency, 17α-hydroxylase deficiency, 3-β-hydroxysteroid dehydrogenase deficiency, congenital lipoid adrenal hyperplasia and p450 oxidoreductase deficiency which all present different symptoms. CAH patients need adequate care and treatment in order to lead normal lives.

### Corticotropin Releasing Factor

Corticotropin releasing factor (CRF) is a 41 amino acid peptide that is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in the brain. There is also evidence that CRF plays a significant role in integrating the response in the immune system to physiological, psychological, and immunological stressors.

CRF has been implicated in psychiatric disorders and neurological diseases including depression and anxiety, as well as the following: Alzheimer's disease, Huntington's disease, progressive supranuclear palsy, amyotrophic lateral sclerosis, Parkinson's disease, epilepsy, migraine, alcohol and substance abuse and associated withdrawal symptoms, obesity, metabolic syndrome, congenital adrenal hyperplasia (CAH), Cushing's disease, hypertension, stroke, irritable bowel syndrome, stress-induced gastric ulceration, premenstrual syndrome, sexual dysfunction, premature labor, inflammatory disorders, allergies, multiple sclerosis, visceral pain, sleep disorders, pituitary tumors or ectopic pituitary derived tumors, chronic fatigue syndrome, and fibromyalgia.

CRF receptor subtypes, CRF1 and CRF2, have been identified and are distributed heterogeneously within the brain thereby suggesting potential functional diversity. For example, widely distributed brain CRF1 receptors are strongly implicated in emotionality accompanying exposure to environmental stressors. Significantly, CRF1, not CRF2, receptors appear to mediate select anxiogenic like behaviors.

Treatment of CAH is based on normalization of hormone and steroid levels using a variety of chronic medications from diagnosis in infancy through adulthood. Glucocorticoids are the current standard treatment in CAH and are used both to correct the endogenous Cortisol deficiency and for reducing the elevated ACTH levels from the pituitary, which drives increased androgen production. Unlike the treatment of Addison's disease (adrenal insufficiency), in which Cortisol replacement is sufficient, the treatment of CAH must also reduce ACTH production, to control the subsequent androgen excess as well. Thus, the goals of glucocorticoid treatment include cortisol replacement and suppression of ACTH to prevent virilization and menstrual disturbances in women. Mineralocorticoid replacement is needed to achieve normal plasma renin activity for maintenance of regular blood pressure, electrolyte balance, and volume status in those patients with the salt-wasting form of CAH.

The regimen of glucocorticoid treatment must support normal physiology and also ensure that sufficient cortisol is available during events that may elicit a strong stress response (e.g., intercurrent illness, exercise, hypotension). Careful monitoring is also necessary to avoid the development of Addisonian syndrome due to under-treatment. Overtreatment with mineralocorticoids may cause hypertension while under-treatment may lead to low blood pressure, salt loss, fatigue and increased requirements for glucocorticoids. Typical laboratory tests for monitoring treatment efficacy include measurement of plasma concentrations of 17-OHP, androstenedione (A4), testosterone, renin activity, and electrolytes.

Adult patients with CAH have an increased prevalence of risk factors for cardiovascular disease including obesity, hypertension, absence of the nadir drop in systolic blood pressure overnight, and insulin resistance. A study of a large cohort of pediatric and adult CAH patients (n=244) demonstrated that patients are prescribed a variety of glucocorticoid treatment regimens yet frequently suffer from poor hormonal control and the aforementioned adverse outcomes. Treatment of CAH includes efforts to normalize the Cortisol deficiency with glucocorticoids (usually hydrocortisone in children and adults). In some embodiments, there is a need for longer acting and more potent agents with narrow therapeutic indices, such as dexamethasone or prednisolone/prednisone, in adults and, if necessary for salt-wasting, mineralocorticoids (usually fludrocortisone). The glucocorticoid doses required to achieve sufficient suppression of excess androgens, however, are usually well above the normal physiologic dose used for cortisol replacement alone as in patients with Addison's disease. This increased exposure to glucocorticoids can lead to increased cardiovascular risk factors, glucose intolerance, and decreased bone mineral density in CAH patients.

CRF is believed to be the major physiological regulator of the basal and stress-induced release of adrenocorticotropic hormone (“ACTH”), β-endorphin, and other proopiomelanocortin (“POMC”)-derived peptides from the anterior pituitary. Secretion of CRF causes release of ACTH from corticotrophs in the anterior pituitary via binding to the CRF1 receptor, a member of the class B family of G-protein coupled receptors.

Due to the physiological significance of CRF1, the development of biologically-active small molecules having significant CRF receptor binding activity and which are capable of antagonizing the CRF1 receptor remains a desirable goal and has been the subject of ongoing research and development for the treatment of anxiety, depression, irritable bowel syndrome, post-traumatic stress disorder, and substance abuse.

The pituitary hormone ACTH, under the control of hypothalamic corticotropin-releasing factor (CRF), stimulates uptake of cholesterol and drives the synthesis of pregnenolone initiating steroidogenesis in the adrenal gland. The adrenal cortex is comprised of three zones, which produce distinct classes of hormones many of which are driven by ACTH mobilizing cholesterol through this pathway. Deficiencies in these enzymes as a result of mutation or deletion cause the substrate concentrations to increase. In the most common form of CAH resulting from mutations or deletions in the 21-hydroxylase gene (CYP21A2), potent androgens are produced by the adrenal because of the accumulation of the steroid precursors, progesterone and 17-OHP. Plasma levels of 17-OHP can reach 10-1000 times the normal concentration in these cases. These increases result in the overproduction of androgens, specifically androstenedione (A4), testosterone, and dihydroxytestosterone causing virilization in females. In addition, 21-hydroxylase deficiency in CAH causes insufficient biosynthesis of glucocorticoids and mineralocorticoids, specifically cortisol and aldosterone. Cortisol is a critical negative feedback regulator of hypothalamic CRF secretion and pituitary ACTH release. The lack of glucocorticoid synthesis and release eliminates the restraint on the hypothalamus and pituitary, which causes ACTH levels to increase. The excessive ACTH stimulation causes hypertrophy of the zona fasciculata and zona reticularis resulting in adrenal hyperplasia.

### Certain Definitions

Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein and in the appended claims, the singular forms “a,” “an,” and “the” include plural reference unless the context clearly dictates otherwise. Thus, for example, reference to “an excipient” is a reference to one or more excipients and equivalents thereof known to those skilled in the art, and so forth.

The term “about” is used to indicate that a value includes 10% level of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and to “and/or.”

“Alkyl” refers to a straight or branched hydrocarbon chain radical consisting solely of carbon and hydrogen atoms, containing no unsaturation, and preferably having from one to five carbon atoms (i.e. C₁-C₅alkyl). In some embodiments, an alkyl comprises one to four carbon atoms (i.e., C₁-C₄alkyl)). In some embodiments, an alkyl comprises one to three carbon atoms (i.e., C₁-C₃alkyl)). In some embodiments, an alkyl comprises one to two carbon atoms (i.e., C₁-C₂alkyl). In some embodiments, an alkyl comprises one carbon atom (i.e., C1alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (isobutyl), 1,1-dimethylethyl (tert-butyl), or 1-pentyl (n-pentyl). The alkyl is attached to the rest of the molecule by a single bond. Unless stated otherwise specifically in the specification, an alkyl group is optionally substituted by one or more substituents such as those described herein.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. “Administering” a pharmaceutical composition may be accomplished by injection, topical administration, and oral administration or by other methods alone or in combination with other known techniques.

By “pharmaceutically acceptable”, it is meant the carrier, diluent or excipient must be compatible with the other ingredients of the composition and not deleterious to the recipient thereof.

The term “pharmaceutical composition” means a composition comprising at least one active ingredient, such as a steroid or a pharmaceutically acceptable salt thereof or a CRF1 antagonist or a pharmaceutically acceptable salt thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “supraphysiologic amount” describes glucocorticoid dose levels that are above the daily glucocorticoid requirement (production rate) found in healthy individuals.

The term “physiologic amount” describes glucocorticoid dose levels that meet the daily glucocorticoid requirement (production rate) found in healthy individuals.

The term “hydrocortisone equivalents” as used herein is understood by a person skilled in the art to be the conversion calculations needed to be considered when substituting one glucocorticoid for another as the potency and duration of action of various glucocorticoids may vary. Hence, the term “hydrocortisone equivalents” is the standard used for comparison of glucocorticoid potency.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

### Compounds

Disclosed herein are CRF1 antagonists or pharmaceutically acceptable salt thereof such as Antalarmin hydrochloride, Pfizer CP 154526, CP 376395 hydrochloride, NBI 27914 hydrochloride, NBI 35965 hydrochloride, NGD 98-2 hydrochloride, Pexacerfont, R 121919 hydrochloride, SNO03, and SSR125543.

In one aspect, the CRF1 antagonist or pharmaceutically acceptable salt thereof is selected from the group consisting of: N-Butyl-N-ethyl-2,5,6-trimethyl-7-(2,4,6-trimethylphenyl)-7H-pyrrolo[2,3-d]pyrimidin-4-amine hydrochloride (Antalarmin hydrochloride), N-Butyl-N-ethyl-2,5-dimethyl-7-(2,4,6-trimethylphenyl)-7H-pyrrolo[2,3-d]pyrimidin-4-amine hydrochloride (Pfizer CP154526), N-(1-Ethylpropyl)-3,6-dimethyl-2-(2,4,6-trimethylphenoxy)-4-pyridinamine hydrochloride (Pfizer CP376395 hydrochloride), 5-Chloro-N-(cyclopropylmethyl)-2-methyl-N-propyl-N′-(2,4,6-trichlorophenyl)-4,6-pyrimidinediamine hydrochloride (NBI27914 hydrochloride), (7S)-6-(Cyclopropylmethyl)-2-(2,4-dichlorophenyl)-7-ethyl-7,8-dihydro-4-methyl-6H-1,3,6,8a-tetraazaacenaphthylene hydrochloride (NBI35965 hydrochloride), N-(1-Ethylpropyl)-3-methoxy-5-[2-methoxy-4-(trifluoromethoxy)phenyl]-6-methyl-2-pyrazinamine hydrochloride (NGD 98-2 hydrochloride), 8-(6-Methoxy-2-methyl-3-pyridinyl)-2,7-dimethyl-N-[(1R)-1-methylpropyl]pyrazolo[1,5-a]-1,3,5-triazin-4-amine (Pexacerfont), 3-[6-(Dimethylamino)-4-methyl-3-pyridinyl]-2,5-dimethyl-N,N-dipropylpyrazolo[1,5-a]pyrimidin-7-amine hydrochloride (R 121919 hydrochloride), and N-(4-Methoxy-2-methylphenyl)-1-[1-(methoxymethyl)propyl]-6-methyl-1H-1,2,3-triazolo[4,5-c]pyridine-4-amine (SNOW), (S)-4-(2-chloro-4-methoxy-5-methylphenyl)-N-(2-cyclopropyl-1-(3-fluoro-4-methylphenyl)ethyl)-5-methyl-N-(prop-2-yn-1-yl)thiazol-2-amine (SSR125543).

Disclosed herein is a compound of Formula (I):

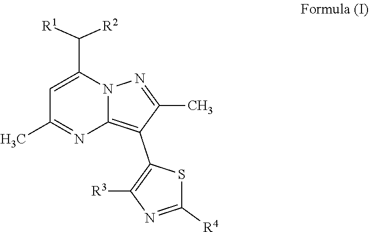

or a pharmaceutically acceptable salt thereof, wherein

R1 and R2 are independently ethyl or n-propyl;

R3 is H, Cl, Br, methyl, trifluoromethyl or methoxy;

R4 is H, Br, RaRbN—, methoxymethyl, n-butyl, acetamido, pyridin-4-yl, morpholin-4-yl,

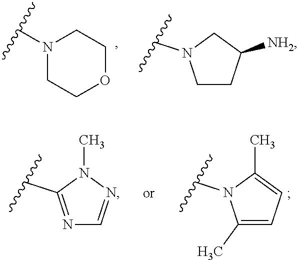

and

Ra and Rb are independently hydrogen, C1-C3 alkyl, H2NCH2CH2-, (CH3)3COC(O)NHCH2CH2-, or CH3CH2CH2NHCH2CH2.

In some embodiments, R3 is hydrogen, Cl, Br, methyl, or trifluoromethyl. In some embodiments, R3 is hydrogen, Cl, Br, or methyl. In some embodiments, R3 is hydrogen, Cl, or Br. In some embodiments, R3 is hydrogen. In some embodiments, R3 is Cl. In some embodiments, R3 is Br.

In some embodiments, R4 is hydrogen, Br, RaRbN—, methoxymethyl, n-butyl, acetamido, pyridin-4-yl, morpholin-4-yl,

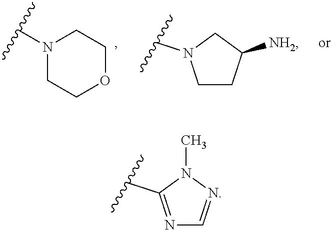

In some embodiments, R4 is Br, RaRbN—, methoxymethyl, n-butyl, acetamido, pyridin-4-yl, morpholin-4-yl,

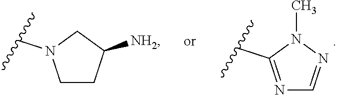

In some embodiments, R4 is RaRbN—, methoxymethyl, n-butyl, acetamido, pyridin-4-yl, morpholin-4-yl, or

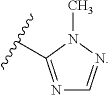 [SMILES: *c1ncnn1C]

In some embodiments, R4 is RaRbN—, n-butyl, acetamido, pyridin-4-yl, morpholin-4-yl, or

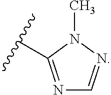 [SMILES: *c1ncnn1C]

In some embodiments, R4 is RaRbN—,pyridin-4-yl, morpholin-4-yl, or

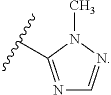 [SMILES: *c1ncnn1C]

In some embodiments, R4 is morpholin-4-yl or

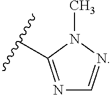 [SMILES: *c1ncnn1C]

In some embodiments, R4 is RaRbN— and Ra and Rb are independently C₁-C₃alkyl.

Disclosed herein is a compound of Formula (II):

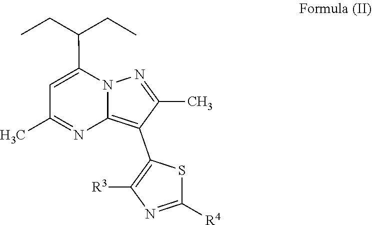

or a pharmaceutically acceptable salt thereof, wherein

R3 is H, Cl, Br, methyl, trifluoromethyl or methoxy;

R4 is H, Br, RaRbN—, methoxymethyl, n-butyl, acetamido, pyridin-4-yl, morpholin-4-yl,

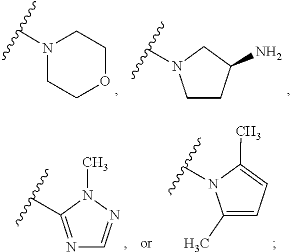

and

Ra and Rb are independently hydrogen, C1-C3 alkyl, H2NCH2CH2-, (CH3)3COC(O)NHCH2CH2-, or CH3CH2CH2NHCH2CH2.

Disclosed herein is a compound of Formula (III):

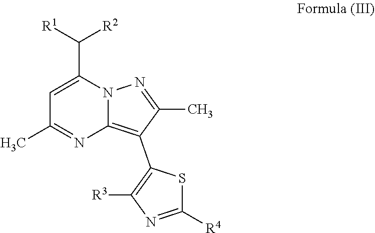

or a pharmaceutically acceptable salt thereof, wherein

R1 and R2 are n-propyl;

R3 is H, Cl, Br, methyl, trifluoromethyl or methoxy;

R4 is H, Br, RaRbN—, methoxymethyl, n-butyl, acetamido, pyridin-4-yl, morpholin-4-yl,

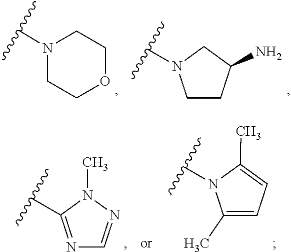

and

Ra and Rb are independently hydrogen, C1-C3 alkyl, H2NCH2CH2-, (CH3)3COC(O)NHCH2CH2-, or CH3CH2CH2NHCH2CH2.

Disclosed herein is a compound of Formula (IV):

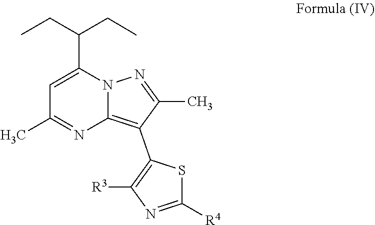

or a pharmaceutically acceptable salt thereof, wherein

R3 is Cl, Br, methyl, trifluoromethyl or methoxy;

R4 is H, Br, RaRbN—, methoxymethyl, n-butyl, acetamido, pyridin-4-yl, morpholin-4-yl,

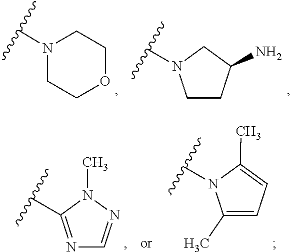

and

Ra and Rb are independently hydrogen, C1-C3 alkyl, H2NCH2CH2-, (CH3)3COC(O)NHCH2CH2-, or CH3CH2CH2NHCH2CH2.

For a compound or pharmaceutically acceptable salt of Formula (I), (II), (III), and (IV), R3 may be hydrogen, Cl, Br, methyl, trifluoromethyl, or methoxy. For a compound or pharmaceutically acceptable salt of Formula (I), (II), (III), and (IV), R3 may be Cl, Br, methyl, trifluoromethyl, or methoxy. For a compound or pharmaceutically acceptable salt of Formula (I), (II), (III), and (IV), R3 may be Cl, Br, methyl, or trifluoromethyl. For a compound or pharmaceutically acceptable salt of Formula (I), (II), (III), and (IV), R3 may be Cl, Br, or methyl. For a compound or pharmaceutically acceptable salt of Formula (I), (II), (III), and (IV), R3 may be Cl. For a compound or pharmaceutically acceptable salt of Formula (I), (II), (III), and (IV), R3 may be Br. For a compound or pharmaceutically acceptable salt of Formula (I), (II), (III), and (IV), R3 may be methyl.

For a compound or pharmaceutically acceptable salt of Formula (I), (II), (III), and (IV), R4 is Br, RaRbN—, methoxymethyl, n-butyl, acetamido, pyridin-4-yl, morpholin-4-yl,

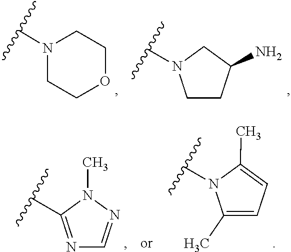

For a compound or pharmaceutically acceptable salt of Formula (I), (II), (III), and (IV), R4 is Br, methoxymethyl, n-butyl, acetamido, pyridin-4-yl, morpholin-4-yl,

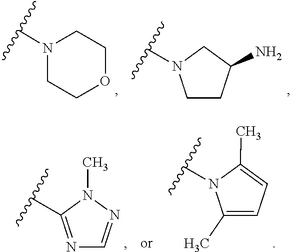

For a compound or pharmaceutically acceptable salt of Formula (I), (II), (III), and (IV), R4 is Br, n-butyl, acetamido, pyridin-4-yl, morpholin-4-yl,

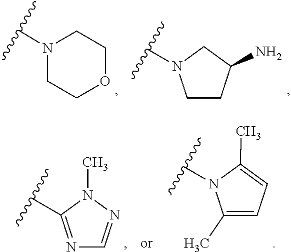

For a compound or pharmaceutically acceptable salt of Formula (I), (II), (III), and (IV), R4 is Br, acetamido, pyridin-4-yl, morpholin-4-yl,

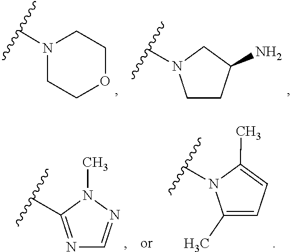

For a compound or pharmaceutically acceptable salt of Formula (I), (II), (III), and (IV), R4 is Br, pyridin-4-yl, morpholin-4-yl,

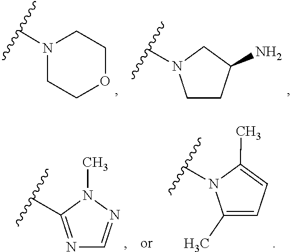

For a compound or pharmaceutically acceptable salt of Formula (I), (II), (III), and (IV), R4 is pyridin-4-yl, morpholin-4-yl,

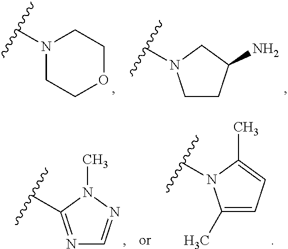

For a compound or pharmaceutically acceptable salt of Formula (I), (II), (III), and (IV), R4 is morpholin-4-yl,

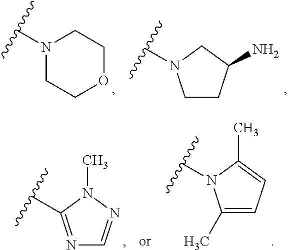

For a compound or pharmaceutically acceptable salt of Formula (I), (II), (III), and (IV), R4 is

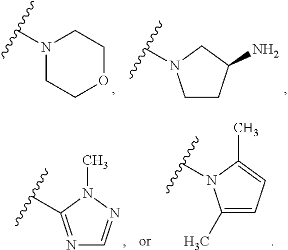

For a compound or pharmaceutically acceptable salt of Formula (I), (II), (III), and (IV), R4 is

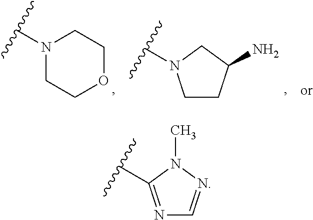

For a compound or pharmaceutically acceptable salt of Formula (I), (II), (III), and (IV), R4 is

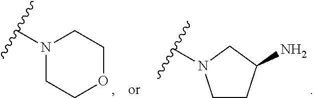 [SMILES: *N1CCOCC1 ; *N1CC[C@H](N)C1]

For a compound or pharmaceutically acceptable salt of Formula (I), (II), (III), and (IV), R4 is

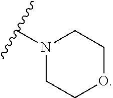 [SMILES: *N1CCOCC1]

Disclosed herein is 3-[4-bromo-2-(2-methyl-2H-[1,2,4]triazol-3-yl)-thiazol-5-yl]-2,5-dimethyl-7-(1-propyl-butyl)-pyrazolo[1,5-a]pyrimidine or a pharmaceutically acceptable salt thereof:

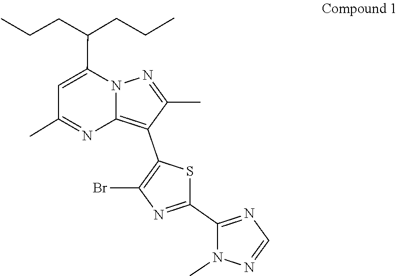 [SMILES: CCCC(CCC)c1cc(C)nc2c(-c3sc(-c4ncnn4C)nc3Br)c(C)nn12]

Disclosed herein is 3-(4-bromo-2-(2-methyl-2H-[1,2,4]triazol-3-yl)-thiazol-5-yl)-7-(1-ethyl-propyl)-2,5-dimethyl-pyrazolo[1,5-a]pyrimidine or a pharmaceutically acceptable salt thereof:

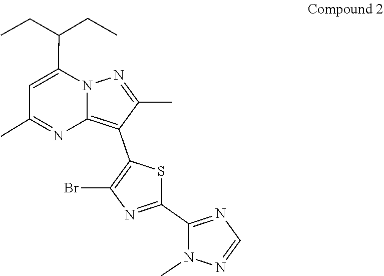 [SMILES: CCC(CC)c1cc(C)nc2c(-c3sc(-c4ncnn4C)nc3Br)c(C)nn12]

Disclosed herein is 3-(4-Chloro-2-(morpholin-4-yl)thiazol-5-yl)-7-(1-ethylpropyl)-2,5-dimethylpyrazolo(1,5-a)pyrimidine (or alternatively 4-(4-chloro-5-(2,5-dimethyl-7-(pentan-3-yl)pyrazolo[1,5-a]pyrimidin-3-yl)thiazol-2-yl)morpholine), a pharmaceutically acceptable salt thereof:

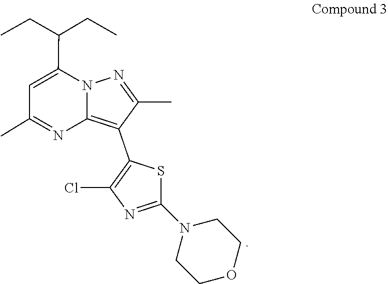 [SMILES: CCC(CC)c1cc(C)nc2c(-c3sc(N4CCOCC4)nc3Cl)c(C)nn12]

In some embodiments, 4-(4-chloro-5-(2,5-dimethyl-7-(pentan-3-yl)pyrazolo[1,5-a]pyrimidin-3-yl)thiazol-2-yl)morpholine is referred to as Compound 3. In some embodiments, 3-(4-Chloro-2-(morpholin-4-yl)thiazol-5-yl)-7-(1-ethylpropyl)-2,5-dimethylpyrazolo(1,5-a)pyrimidine is referred to as Compound 3.

In one aspect, the CRF1 antagonist or pharmaceutically acceptable salt thereof may be an astressin. An astressin generally refers to a nonselective corticotropin releasing hormone antagonist that reduces the synthesis of ACTH and cortisol.

### Pharmaceutical Compositions

Disclosed herein are pharmaceutical compositions comprising a compound or pharmaceutically acceptable salt described herein. In certain embodiments, the composition comprises a steroid or a pharmaceutically acceptable salt thereof and a CRF1 antagonist or pharmaceutically acceptable salt thereof as disclosed above. In some embodiments, the steroid or a pharmaceutically acceptable salt thereof is exogenous glucocorticoids (GC) or a pharmaceutically acceptable salt thereof.

### Dosage Form

In some embodiments, the pharmaceutical compositions described herein are provided in unit dosage form. As used herein, a “unit dosage form” is a composition containing an amount of a compound or pharmaceutically acceptable salt described herein that is suitable for administration to an animal, preferably mammal, subject in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical compositions described herein are formulated as oral dosage forms. Suitable oral dosage forms include, for example, tablets, pills, sachets, or capsules. In some embodiments, the pharmaceutical composition comprises one or more additional pharmaceutically acceptable excipients. See, e.g., Remington: The Science and Practice of Pharmacy (Gennaro, 21st Ed. Mack Pub. Co., Easton, Pa. (2005) for a list of pharmaceutically acceptable excipients.

### Capsule

In some embodiments, the pharmaceutical composition is formulated as a capsule. In some embodiments, the pharmaceutical composition is formulated as a hard gel capsule. In some embodiments, the pharmaceutical composition is formulated as a soft gel capsule.

In some embodiments, the capsule is formed using materials which include, but are not limited to, natural or synthetic gelatin, pectin, casein, collagen, protein, modified starch, polyvinylpyrrolidone, acrylic polymers, cellulose derivatives, or any combinations thereof. In some embodiments, the capsule is formed using preservatives, coloring and opacifying agents, flavorings and sweeteners, sugars, gastroresistant substances, or any combinations thereof. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the capsule is broken such that the particulates are sprinkled on soft foods and swallowed without chewing. In some embodiments, the shape and size of the capsule also vary. Examples of capsule shapes include, but are not limited to, round, oval, tubular, oblong, twist off, or a non-standard shape. The size of the capsule may vary according to the volume of the particulates. In some embodiments, the size of the capsule is adjusted based on the volume of the particulates and powders. Hard or soft gelatin capsules may be manufactured in accordance with conventional methods as a single body unit comprising the standard capsule shape. A single-body soft gelatin capsule typically may be provided, for example, in sizes from 3 to 22 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size. In some embodiments, the pharmaceutical composition described herein (e.g., capsule) is swallowed as a whole.

In some embodiments, the capsule comprises one or more pharmaceutically acceptable excipients. In some embodiments, the capsule is free of additional excipients.

In some embodiments, a capsule is developed, manufactured and commercialized for a drug substance that is insoluble. In some embodiments, a drug substance is insoluble if solubility is less than 0.002 mg/mL in water. In some embodiments, the capsule has a dose strength of up to 200 mg. In some embodiments, drug substance in the capsule is immediately released in a dissolution medium using USP apparatus I. In some embodiments, drug substance in the capsule is immediately released in a dissolution medium using USP apparatus II.

### Tablet

Poorly soluble drugs may be difficult to formulate using standard technologies such as high shear wet granulation. Optimum delivery of poorly soluble drugs may require complex technologies such as solid solutions amorphous dispersions (hot melt extrusion or spray drying), nano-formulations or lipid-based formulations. Hydrophobic drug substances may be considered poorly soluble according to USP criteria and may be known to be difficult to granulate with water and other excipients. This is likely due to most known excipients for immediate release formulations being water soluble or water-swellable. Making a tablet of a high dose drug substance that is poorly soluble may require a high concentration of the drug substance. However, as the drug concentration is increased above a certain level, formation of granules may become more and more difficult. Furthermore, at a certain drug load, it may become impossible.

In some embodiments, the pharmaceutical composition is formulated as a tablet.

In some embodiments, the tablet is made by compression, molding, or extrusion, optionally with one or more pharmaceutically acceptable excipient. In some embodiments, compressed tablets are prepared by compressing a compound or pharmaceutically acceptable salt described herein in a free-flowing form, optionally mixed with pharmaceutically acceptable excipients. In some embodiments, molded tablets are made by molding a mixture of the powdered a compound or pharmaceutically acceptable salt described herein, moistened with an inert liquid diluent. In some embodiments, the tablet is prepared by hot-melt extrusion. In some embodiments, extruded tablets are made by forcing a mixture comprising a compound or pharmaceutically acceptable salt described herein, through an orifice or die under controlled conditions. In some embodiments, the tablet is coated or scored. In some embodiments, the tablet is formulated so as to provide slow or controlled release of a compound or pharmaceutically acceptable salt described herein. In some embodiments, a tablet is developed, manufactured and commercialized for a drug substance that is insoluble. In some embodiments, a drug substance is insoluble if solubility is less than 0.002 mg/mL in water. In some embodiments, the tablet has a dose strength of up to 200 mg. In some embodiments, drug substance in the tablet is immediately released in a dissolution medium using USP apparatus I. In some embodiments, drug substance in the tablet is immediately released in a dissolution medium using USP apparatus II.

In some embodiments, the tablet size is less than about 1000 mg, less than about 800 mg, less than about 600 mg, less than about 400 mg or less than about 200 mg. In some embodiments, the tablet has a dose strength of more than about 50 mg, more than about 100 mg, more than about 150 mg, more than about 200 mg, or more than about 250 mg. In some embodiments, the tablet size is less than about 1000 mg for a dose strength of more than about 50 mg. In some embodiments, the tablet size is less than 800 mg for a dose strength of more than about 100 mg. In some embodiments, the tablet size is less than 600 mg for a dose strength of more than about 150 mg. In some embodiment, the tablet size is less than 400 mg for a dose strength of more than about 200 mg. In some embodiments, the tablet size is less than 400 mg for a dose strength of 200 mg.

In some embodiments, more than about 20% of the tablet is dissolved in conventional dissolution media. In some embodiments, more than about 40% of the tablet is dissolved in conventional dissolution media. In some embodiments, more than about 50% of the tablet is dissolved in conventional dissolution media. In some embodiments, more than about 60% of the tablet is dissolved in conventional dissolution media. In some embodiments, more than about 70% of the tablet is dissolved in conventional dissolution media. In some embodiments, more than about 80% of the tablet is dissolved in conventional dissolution media. In some embodiments, more than about 20% of the tablet is dissolved in less than 24 hours in conventional dissolution media. In some embodiments, more than about 20% of the tablet is dissolved in less than 12 hours in conventional dissolution media. In some embodiments, more than about 20% of the tablet is dissolved in less than 6 hours in conventional dissolution media. In some embodiments, more than about 20% of the tablet is dissolved in less than 3 hours in conventional dissolution media. In some embodiments, more than about 20% of the tablet is dissolved in less than 2 hours in conventional dissolution media. In some embodiments, more than about 20% of the tablet is dissolved in less than 60 minutes in conventional dissolution media. In some embodiments, more than about 40% of the tablet is dissolved in less than 60 minutes in conventional dissolution media. In some embodiments, more than about 50% of the tablet is dissolved in less than 60 minutes in conventional dissolution media. In some embodiments, more than about 60% of the tablet is dissolved in less than 60 minutes in conventional dissolution media. In some embodiments, more than about 70% of the tablet is dissolved in less than 60 minutes in conventional dissolution media. In some embodiments, more than about 80% of the tablet is dissolved in less than 60 minutes in conventional dissolution media. In some embodiments, more than 70% of the tablet is dissolved in 60 minutes in conventional dissolution media.

In some embodiments, the tablet is produced at a commercial scale.

In some embodiments, the tablet comprises one or more pharmaceutically acceptable excipients.

In some embodiments, the tablet is coated with a coating material, e.g., a sealant. In some embodiments, the coating material is water soluble. In some embodiments, the coating material comprises a polymer, plasticizer, a pigment, or any combination thereof. In some embodiments, the coating material is in the form of a film coating, e.g., a glossy film, a pH independent film coating, an aqueous film coating, a dry powder film coating (e.g., complete dry powder film coating), or any combination thereof. In some embodiments, the coating material is highly adhesive. In some embodiments, the coating material provides low level of water permeation. In some embodiments, the coating material provides oxygen barrier protection. In some embodiments, the coating material allows immediate disintegration for fast release of a compound or pharmaceutically acceptable salt described herein. In some embodiments, the coating material is pigmented, clear, or white. In some embodiments, the coating is an enteric coating. Exemplary coating materials include, without limitation, polyvinylpyrrolidone, polyvinyl alcohol, an acrylate-methacrylic acid copolymer, a methacrylate-methacrylic acid copolymer, cellulose acetate phthalate, cellulose acetate succinate, hydroxypropyl methylcellulose phthalate, hydroxypropyl methylcellulose acetate succinate, polyvinyl acetate phthalate, shellac, cellulose acetate trimellitate, sodium alginate, zein, and any combinations thereof.

### Pharmaceutically Acceptable Excipients

In some embodiments, the pharmaceutical composition comprises a pharmaceutically acceptable excipient. In some embodiments, the composition is free of pharmaceutically acceptable excipients. The term “pharmaceutically acceptable excipient”, as used herein, means one or more compatible solid or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. In some embodiments, the pharmaceutically acceptable excipient is of sufficiently high purity and sufficiently low toxicity to render them suitable for administration preferably to an animal, preferably mammal, being treated.

Some examples of substances, which can serve as pharmaceutically acceptable excipients include:
Amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the amino acid is arginine. In some embodiments, the amino acid is L-arginine.
Monosaccharides such as glucose (dextrose), arabinose, mannitol, fructose (levulose), and galactose.
Cellulose and its derivatives such as sodium carboxymethyl cellulose, ethyl cellulose, and methyl cellulose.
Solid lubricants such as talc, stearic acid, magnesium stearate, and sodium stearyl fumarate.
Polyols such as propylene glycol, glycerin, sorbitol, mannitol, and polyethylene glycol.
Emulsifiers such as the polysorbates.
Wetting agents such as sodium lauryl sulfate, Tween, Span, alkyl sulphates, and alkyl ethoxylate sulphates.
Diluents such as calcium carbonate, microcrystalline cellulose, calcium phosphate, starch, pregelatinized starch, sodium carbonate, mannitol, and lactose.
Binders such as starches (corn starch and potato starch), gelatin, sucrose, hydroxypropyl cellulose (HPC), polyvinylpyrrolidone (PVP), and hydroxypropyl methyl cellulose (HPMC).
Disintegrants such as starch, and alginic acid.
Super-disintegrants such as ac-di-sol, croscarmellose sodium, sodium starch glycolate and crospovidone.
Glidants such as silicon dioxide.
Coloring agents such as the FD&C dyes.
Sweeteners and flavoring agents, such as aspartame, saccharin, menthol, peppermint, and fruit flavors.
Preservatives such as benzalkonium chloride, PHMB, chlorobutanol, thimerosal, phenylmercuric, acetate, phenylmercuric nitrate, parabens, and sodium benzoate.
Tonicity adjustors such as sodium chloride, potassium chloride, mannitol, and glycerin.
Antioxidants such as sodium bisulfite, acetone sodium bisulfite, sodium formaldehyde, sulfoxylate, thiourea, and EDTA.
pH adjuster such as NaOH, sodium carbonate, sodium acetate, HCl, and citric acid.
Cryoprotectants such as sodium or potassium phosphates, citric acid, tartaric acid, gelatin, and carbohydrates such as dextrose, mannitol, and dextran.
Cationic surfactants such as cetrimide, benzalkonium chloride and cetylpyridinium chloride.
Anion surfactants such as alkyl sulphates, alkyl ethoxylate sulphates, soaps, carboxylates, sulfates, and sulfonates.
Non-ionic surfactants such as polyoxyethylene derivatives, polyoxypropylene derivatives, polyol derivatives, polyol esters, polyoxyethylene esters, poloxamers, glyol esters, glycerol esters, sorbitan derivatives, polyethylene glycol (PEG-40, PEG-50, PEG-55), and ethers of fatty alcohols.
Organic materials such as carbohydrate and modified carbohydrates, lactose, a-lactose monohydrate, spray dried lactose and anhydrous lactose, starch and pre-gelatinized starch, sucrose, manitol, sorbitol, cellulose, powdered cellulose and microcrystalline cellulose.
Inorganic materials such as calcium phosphates (anhydrous dibasic calcium phosphate, dibasic calcium phosphate and tribasic calcium phosphate).
Co-processed diluents.
Surfactants such as sodium lauryl sulfate.
Compression aids.
Anti-tacking agents such as silicon dioxide and talc

### Amounts

In some embodiments, the pharmaceutical composition, in the form of a tablet or capsule, comprises between about 1 mg and about 500 mg of a compound or pharmaceutically acceptable salt described herein. In some embodiments, the pharmaceutical composition, in the form of a tablet or capsule, comprises between about 1 mg and about 400 mg of a compound or pharmaceutically acceptable salt described herein. In some embodiments, the pharmaceutical composition, in the form of a tablet or capsule, comprises between about 1 mg and about 300 mg of a compound or pharmaceutically acceptable salt described herein. In some embodiments, the pharmaceutical composition, in the form of a tablet or capsule, comprises between about 1 mg and about 200 mg of a compound or pharmaceutically acceptable salt described herein. In some embodiments, the pharmaceutical composition, in the form of a tablet or capsule, comprises between about 1 mg and about 100 mg of a compound or pharmaceutically acceptable salt described herein. In some embodiments, the pharmaceutical composition, in the form of a tablet or capsule, comprises between about 1 mg and about 90 mg of a compound or pharmaceutically acceptable salt described herein. In some embodiments, the pharmaceutical composition, in the form of a tablet or capsule, comprises between about 1 mg and about 80 mg of a compound or pharmaceutically acceptable salt described herein. In some embodiments, the pharmaceutical composition, in the form of a tablet or capsule, comprises between about 1 mg and about 70 mg of a compound or pharmaceutically acceptable salt described herein. In some embodiments, the pharmaceutical composition, in the form of a tablet or capsule, comprises between about 1 mg and about 60 mg of a compound or pharmaceutically acceptable salt described herein. In some embodiments, the pharmaceutical composition, in the form of a tablet or capsule, comprises between about 1 mg and about 50 mg of a compound or pharmaceutically acceptable salt described herein. In some embodiments, the pharmaceutical composition, in the form of a tablet or capsule, comprises between about 1 mg and about 40 mg of a compound or pharmaceutically acceptable salt described herein. In some embodiments, the pharmaceutical composition, in the form of a tablet or capsule, comprises between about 1 mg and about 30 mg of a compound or pharmaceutically acceptable salt described herein. In some embodiments, the pharmaceutical composition, in the form of a tablet or capsule, comprises between about 1 mg and about 20 mg of a compound or pharmaceutically acceptable salt described herein. In some embodiments, the pharmaceutical composition, in the form of a tablet or capsule, comprises between about 1 mg and about 10 mg of a compound or pharmaceutically acceptable salt described herein.

In some embodiments, the pharmaceutical composition, in the form of a tablet or capsule, comprises between about 10 mg and about 500 mg of a compound or pharmaceutically acceptable salt described herein. In some embodiments, the pharmaceutical composition, in the form of a tablet or capsule, comprises between about 10 mg and about 400 mg of a compound or pharmaceutically acceptable salt described herein. In some embodiments, the pharmaceutical composition, in the form of a tablet or capsule, comprises between about 10 mg and about 300 mg of a compound or pharmaceutically acceptable salt described herein. In some embodiments, the pharmaceutical composition, in the form of a tablet or capsule, comprises between about 10 mg and about 200 mg of a compound or pharmaceutically acceptable salt described herein. In some embodiments, the pharmaceutical composition, in the form of a tablet or capsule, comprises between about 10 mg and about 100 mg of a compound or pharmaceutically acceptable salt described herein. In some embodiments, the pharmaceutical composition, in the form of a tablet or capsule, comprises between about 10 mg and about 90 mg of a compound or pharmaceutically acceptable salt described herein. In some embodiments, the pharmaceutical composition, in the form of a tablet or capsule, comprises between about 10 mg and about 80 mg of a compound or pharmaceutically acceptable salt described herein. In some embodiments, the pharmaceutical composition, in the form of a tablet or capsule, comprises between about 10 mg and about 70 mg of a compound or pharmaceutically acceptable salt described herein. In some embodiments, the pharmaceutical composition, in the form of a tablet or capsule, comprises between about 10 mg and about 60 mg of a compound or pharmaceutically acceptable salt described herein f. In some embodiments, the pharmaceutical composition, in the form of a tablet or capsule, comprises between about 10 mg and about 50 mg of a compound or pharmaceutically acceptable salt described herein. In some embodiments, the pharmaceutical composition, in the form of a tablet or capsule, comprises between about 10 mg and about 40 mg of a compound or pharmaceutically acceptable salt described herein. In some embodiments, the pharmaceutical composition, in the form of a tablet or capsule, comprises between about 10 mg and about 30 mg of a compound or pharmaceutically acceptable salt described herein. In some embodiments, the pharmaceutical composition, in the form of a tablet or capsule, comprises between about 10 mg and about 20 mg of a compound or pharmaceutically acceptable salt described herein.

### Particle Size

In some embodiments, the pharmaceutical composition, in the form of a tablet or a capsule, comprises a compound or pharmaceutically acceptable salt described herein, in the form of microparticles. In some embodiments, the microparticles have an average size from about 1 μm to about 100 μm. In some embodiments, the microparticles have an average size from about 1 μm to about 50 μm. In some embodiments, the microparticles have an average size from about 1 μm to about 30 μm. In some embodiments, the microparticles have an average size from about 1 μm to about 20 μm. In some embodiments, the microparticles have an average size from about 5 μm to about 15 μm. In some embodiments, the microparticles have an average size from about 1 μm to about 10 μm. In some embodiments, the microparticles have an average size from about 3 μm to about 10 μm. In some embodiments, the microparticles have an average size from about 4 μm to about 9 μm.

### Methods of Treatment

Disclosed herein are methods of treating congenital adrenal hyperplasia (CAH) in a subject in need thereof, comprising administering a compound or pharmaceutically acceptable salt described herein. In some embodiments, the methods described herein result in the reduction of a level of a hormone. Such hormones include deoxycorticosterone, 11-deoxycortisol, cortisol, corticosterone, aldosterone, pregnenolone, 17α-hydroxy pregnenolone, progesterone, 17-OHP, dehydroepiandrosterone, androstenediol, A4, testosterone, dihydrotestosterone, estrone, estradiol, estriol, and ACTH. In some embodiments, the methods described herein result in the reduction of 17-OHP levels. In some embodiments, the methods described herein result in the reduction of A4 levels. In some embodiments, the methods described herein result in the reduction of ACTH levels, also known as corticotropin.

Further, in some embodiments, the methods described herein result in the maintenance of the reduction of 17-OHP, A4, and/or ACTH levels. In some embodiments, the reductions of 17-OHP, A4, and/or ACTH levels may last for at least 24 hours, 36 hours, 48 hours, 60 hours, 72 hours, 4 days, 5 days, 6 days, 7 days, 8 days, 9 days, 10 days, 11 days, 12 days, 13 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 2 years, 5 years, 10 years, 15 years, 20 years, 25 years, 30 years, 35 years, 40 years, 45 years, 50 years, 55 years, 60 years, 65 years, 70 years, 75 years, 80 years, 85 years, 90 years, 95 years, or 100 years.

Moreover, the present disclosure provides different patient subtype regimens for treating Congenital Adrenal Hyperplasia (CAH) patients. In one aspect, the present disclosure provides a method of treating Congenital Adrenal Hyperplasia (CAH), comprising administering an effective amount of a steroid or a pharmaceutically acceptable salt thereof and a corticotropin-releasing factor type-1 (CRF1) antagonist or a pharmaceutically acceptable salt thereof to a subject, i.e., a CAH patient/individual, over a 24 hour period, which the effective amount of the steroid is dependent on the levels of steroid hormones, such as ACTH, A4, 17-OHP, or any combinations thereof, of the subject measured at the time of beginning the treatment of CAH, typically in the am. Prior to implementing the method of treatment disclosed herein, a CAH patient may be evaluated to determine the patient's plasma steroid hormone levels.

Generally, a normal range, a normal laboratory reference range, or a reference range of ACTH level or A4 level in a subject may be broad and dependent on many different factors. For example, ACTH, 17-OHP or A4 is secreted in bursts with a daily rhythm in a subject. Plasma samples collected at 8 am in the morning may show different range of ACTH or A4 levels in a subject compared to plasma samples collected at 2 pm, 4 pm, or midnight during the day. Further, the normal range of ACTH or A4 levels are dependent on a factor such as laboratory setting. Laboratory setting may comprise different types of assays for detecting ACTH or A4 levels, equipment utilized in the laboratory, personnel in the laboratory performing the assays, and different batches of reagents used during the assays. Different ages of a subject may provide different normal ranges of ACTH or A4 levels as well. Moreover, a normal range of ACTH level or A4 level in a subject may be dependent on the sex of the subject. For example, not meant to be limiting, a male subject's normal range of ACTH level may be from 7 to 63.3 pg/mL, as assessed in the am, typically before 10 am, and A4 level, also assessed in the am, typically before 10 am, may be from 50 to 220 ng/dL for males aged 18 to 30 years, from 40 to 190 ng/dL for males aged 31 to 50 years and from 50 to 220 ng/dL for males aged 51 to 60 years. Additionally, a normal range of ACTH level or A4 level in a female subject may be dependent on the female subject's menopausal stage. For example, not meant to be limiting, a pre-menopausal female subject's normal range of ACTH level may be from 7 to 63 pg/mL, as assessed in the am, typically before 10 am, and A4 level may be from 51 to 213 ng/dL for women in the mid-follicular stage, from 73 to 230 ng/dL for women in the surge stage, from 73 to 184 for women in the mid luteal stage. A post-menopausal female subject's normal range of ACTH level may be from 7 to 63.3 pg/mL and A4 level may be from 20 to 75 ng/dL. Normal ranges for PM assessments of ACTH and A4 have not been established.

In some embodiments, when a subject has levels that are less than two (2) times an upper limit of the normal ranges or the normal laboratory reference ranges of ACTH and A4 level respectively, a percentage of the total daily GC dose, typically done in 5 mg hydrocortisone equivalents, of the effective amount of the steroid or a pharmaceutically acceptable salt thereof is reduced compared to a dose of the effective amount of the steroid or a pharmaceutically acceptable salt thereof administered to a CAH patient without receiving a CRF1 antagonist (i.e., a compound disclosed herein) or a pharmaceutically acceptable salt thereof. In some embodiments, the subject has a level that is less than 2 times the upper limit of the normal range of ACTH or A4 level. In some embodiments, the subject has a level that is less than 1.75 times the upper limit of the normal range of ACTH or A4 level. In some embodiments, the subject has a level that is less than 1.5 times the upper limit of the normal range of ACTH or A4 level. In some embodiments, the subject has a level that is less than 1.25 times the upper limit of the normal range of ACTH or A4 level. In some embodiments, the subject has a level that is less than 1.0 times the upper limit of the normal range of ACTH or A4 level.

In certain embodiments, as long as the levels of both the ACTH and A4 are less than 2 times an upper limit of the normal reference ranges of ACTH and A4 respectively, the dosage of the steroid or a pharmaceutically acceptable salt thereof is reduced. For example, a CAH patient's biological sample may be measured to show that the patient's ACTH level is less than 1.75 times the upper limit of the normal range of ACTH and A4 level is less than 1.25 the upper limit of the normal range of A4. Further, a CAH patient's biological sample may be measured to show that the patient's ACTH level is less than 1.25 times the upper limit of the normal range of ACTH and A4 level is less than 1.5 the upper limit of the normal range of A4. Moreover, a CAH patient's biological sample may be measured to show that the patient's ACTH level is less than 1.5 times the upper limit of the normal range of ACTH and A4 level is less than 1.5 the upper limit of the normal range of A4. Additionally, a CAH patient's biological sample may be measured to show that the patient's ACTH level is less than 1.25 times the upper limit of the normal range of ACTH and A4 level is less than 1.25 the upper limit of the normal range of A4. The regimen of treating with reduced amount of the steroid or a pharmaceutically acceptable salt thereof compared to the amount of the steroid or a pharmaceutically acceptable salt thereof administered to a CAH patient without receiving a CRF1 antagonist (i.e., a compound disclosed herein) or a pharmaceutically acceptable salt thereof may be utilized. The reduction of GC daily does may be done in a 5 mg hydrocortisone equivalents, 4 mg hydrocortisone equivalents, 3 mg hydrocortisone equivalents, 2 mg hydrocortisone equivalents, 1 mg hydrocortisone equivalents, or 0.5 mg hydrocortisone equivalents.

In some embodiments, when a subject has levels that are less than two (2) times an upper limit of the normal ranges or the normal laboratory reference ranges of ACTH and 17-OHP level respectively, a percentage of the total daily GC dose, typically done in 5 mg hydrocortisone equivalents, of the effective amount of the steroid or a pharmaceutically acceptable salt thereof is reduced compared to a dose of the effective amount of the steroid or a pharmaceutically acceptable salt thereof administered to a CAH patient without receiving a CRF1 antagonist (i.e., a compound disclosed herein) or a pharmaceutically acceptable salt thereof. In some embodiments, the subject has a level that is less than 2 times the upper limit of the normal range of ACTH or 17-OHP level. In some embodiments, the subject has a level that is less than 1.75 times the upper limit of the normal range of ACTH or 17-OHP level. In some embodiments, the subject has a level that is less than 1.5 times the upper limit of the normal range of ACTH or 17-OHP level. In some embodiments, the subject has a level that is less than 1.25 times the upper limit of the normal range of ACTH or 17-OHP level. In some embodiments, the subject has a level that is less than 1.0 times the upper limit of the normal range of ACTH or 17-OHP level. As long as the levels of both the ACTH and 17-OHP are less than 2 times an upper limit of the normal reference ranges of ACTH and 17-OHP respectively, the dosage of the steroid or a pharmaceutically acceptable salt thereof is reduced. For example, a CAH patient's biological sample may be measured to show that the patient's ACTH level is less than 1.5 times the upper limit of the normal range of ACTH and 17-OHP level is less than 1.75 the upper limit of the normal range of 17-OHP. Further, a CAH patient's biological sample may be measured to show that the patient's ACTH level is less than 1 times the upper limit of the normal range of ACTH and 17-OHP level is less than 1.25 the upper limit of the normal range of 17-OHP. The regimen of treating with reduced amount of the steroid or a pharmaceutically acceptable salt thereof compared to the amount of the steroid or a pharmaceutically acceptable salt thereof administered to a CAH patient without receiving a CRF1 antagonist (i.e., a compound disclosed herein) or a pharmaceutically acceptable salt thereof may be utilized.

In some embodiments, when a subject has levels that are less than two (2) times an upper limit of the normal ranges or the normal laboratory reference ranges of A4 and 17-OHP level respectively, a percentage of the total daily GC dose, typically done in 5 mg hydrocortisone equivalents, of the effective amount of the steroid or a pharmaceutically acceptable salt thereof is reduced compared to a dose of the effective amount of the steroid or a pharmaceutically acceptable salt thereof administered to a CAH patient without receiving a CRF1 antagonist (i.e., a compound disclosed herein) or a pharmaceutically acceptable salt thereof. In some embodiments, the subject has a level that is less than 2 times the upper limit of the normal range of A4 or 17-OHP level. In some embodiments, the subject has a level that is less than 1.75 times the upper limit of the normal range of A4 or 17-OHP level. In some embodiments, the subject has a level that is less than 1.5 times the upper limit of the normal range of A4 or 17-OHP level. In some embodiments, the subject has a level that is less than 1.25 times the upper limit of the normal range of A4 or 17-OHP level. In some embodiments, the subject has a level that is less than 1.0 times the upper limit of the normal range of A4 or 17-OHP level. As long as the levels of both the A4 and 17-OHP are less than 2 times an upper limit of the normal reference ranges of A4 and 17-OHP respectively, the dosage of the steroid or a pharmaceutically acceptable salt thereof is reduced. For example, a CAH patient's biological sample may be measured to show that the patient's A4 level is less than 1 times the upper limit of the normal range of A4 and 17-OHP level is less than 1.75 the upper limit of the normal range of 17-OHP. Further, a CAH patient's biological sample may be measured to show that the patient's A4 level is less than 1.5 times the upper limit of the normal range of A4 and 17-OHP level is less than 1.25 the upper limit of the normal range of 17-OHP. The regimen of treating with reduced amount of the steroid or a pharmaceutically acceptable salt thereof compared to the amount of the steroid or a pharmaceutically acceptable salt thereof administered to a CAH patient without receiving a CRF1 antagonist (i.e., a compound disclosed herein) or a pharmaceutically acceptable salt thereof may be utilized.

When a CRF1 antagonist is not administered, treatment of CAH patients needs to replace the cortisol deficiency and normalize the excess androgen production, which is a sequalae of the enzymatic deficiency in CAH, by administering exogenous glucocorticoids (GC) at a dose that is usually well above the normal physiologic does used for cortisol replacement in patients with other diseases, such as Addison's disease. Reduced levels of exposure to steroids, form supraphysiologic to physiologic levels, may be beneficial to CAH patients. In some embodiments, a dose of about 10 mg to about 80 mg GC is administered to a CAH patient when a CRF1 antagonist is not administered. In some embodiments, the dose of GC is administered to CAH patients one time, two times, three times, or four times daily.

For example, not meant to be limiting, a dose of about 80 mg GC could be administered to a CAH patient when a CRF1 antagonist is not administered. If this CAH patient's ACTH level and A4 level are less than 2, 1.5, or 1.25 times the upper limit of the normal range of ACTH level and A4 levels respectively after controlled by factors such as age, sex, menopausal status, laboratory setting, the dose of 80 mg GC may be reduced in a 5 mg hydrocortisone equivalent increment when administered together with the CRF1 antagonist (i.e., a compound disclosed herein). In some embodiments, a corticotropin-releasing factor type-1 (CRF1) antagonist or a pharmaceutically acceptable salt thereof in a dose of about 10 mg to about 200 mg total daily dose is administered to the subject. The CAH patient may be receiving a dose of 75 mg GC during the treatment regimen when the compound is administered together. After a certain period, when the CAH patient's ACTH level and A4 levels remain less than 2, 1.5, or 1.25 times the upper limit of the normal range of the ACTH and A4 levels respectively, the dose of 75 mg GC may be further reduced in a 5 mg increment to 70 mg when administered with the CRF1 antagonist. The dose of the GC may be continuously reduced in a 5 mg increment hydrocortisone equivalent when administered together with the CRF1 antagonist, over a 24 hour period, until the subject's ACTH or A4 level is not less than 2, 1.5, or 1.25 times the upper limit of the normal range of ACTH or A4 level.

Moreover, if this CAH patient's ACTH level is less than 1.75 times the upper limit of the normal range of ACTH level and A4 level is less than 1.5 times the upper limit of the normal range of A4 level after controlled by factors such as age, sex, menopausal status, laboratory setting, the dose of 80 mg GC may be reduced in a 5 mg increment when administered together the CRF1 antagonist (i.e., a compound disclosed herein). If this CAH patient's ACTH level is less than 1.5 times the upper limit of the normal range of ACTH level and A4 level is less than 1.75 times the upper limit of the normal range of A4 level after controlled by factors such as age, sex, menopausal status, laboratory setting, the dose of 80 mg GC may be reduced in a 5 mg increment when administered together the CRF1 antagonist (i.e., a compound disclosed herein). If this CAH patient's ACTH level is less than 1.25 times the upper limit of the normal range of ACTH level and A4 level is less than 1.25 times the upper limit of the normal range of A4 level after controlled by factors such as age, sex, menopausal status, laboratory setting, the dose of 80 mg GC may be reduced in a 5 mg increment when administered together the CRF1 antagonist (i.e., a compound disclosed herein). In some embodiments, if this CAH patient's ACTH level is less than 1.0 times the upper limit of the normal range of ACTH level and A4 level is less than 1.0 times the upper limit of the normal range of A4 level after controlled by factors such as age, sex, menopausal status, laboratory setting, the dose of 80 mg GC may be reduced in a 5 mg increment when administered together with the CRF1 antagonist (i.e., a compound disclosed herein).

In some embodiments, the subject will be treated for a period of about 1 week to about 40 weeks. In some embodiments, the subject will be treated for a period of about 2 weeks to about 39 weeks. In some embodiments, the subject will be treated for a period of about 3 weeks to about 38 weeks. In some embodiments, the subject will be treated for a period of about 4 weeks to about 36 weeks. In some embodiments, if a subject has been treated for a period of time described herein and has levels of A4 and ACTH less than the lower limit of the normal laboratory reference range, the subject may continue with steroid reduction until the subject maintain hormone levels consistently within the normal range of A4 and ACTH.

Further, in some embodiments, when a subject has a level that is more than an upper limit of the normal range or the normal laboratory reference range of ACTH or A4 level, the total daily dose of the effective amount of the steroid or a pharmaceutically acceptable salt thereof is not reduced compared to a dose of the effective amount of the steroid or a pharmaceutically acceptable salt thereof administered to a CAH patient without receiving a CRF1 antagonist (i.e., a compound disclosed herein) or a pharmaceutically acceptable salt thereof. In some embodiments, the subject has a level that is more than 2 times the upper limit of the normal range of ACTH or A4 level. Some subjects may further be categorized as having severe control issues when the subjects have more than 4 times an upper limit of the normal range of both ACTH and A4 levels. Moreover, some subjects may be further categorized as having moderate control issues when the subjects have more than 2 times the upper limit of normal range of the ACTH level but less than 4 times the upper limit of normal range of the A4 level. In some embodiments, the subject has a level that is more than 1.75 times the upper limit of the normal range of ACTH or A4 level. In some embodiments, the subject has a level that is more than 1.5 times the upper limit of the normal range of ACTH or A4 level. In some embodiments, the subject has a level that is more than 1.25 times the upper limit of the normal range of ACTH or A4 level. In some embodiments, the subject has a level that is more than 1.0 times the upper limit of the normal range of ACTH or A4 level.

Additionally, in some embodiments, when a subject has an ACTH level that is more than an upper limit of the normal range or the normal laboratory reference range of ACTH level and an A4 level that is more than an upper limit of the normal range or the normal laboratory reference range of A4, the total daily dose of the effective amount of the steroid or a pharmaceutically acceptable salt thereof is not reduced compared to a dose of the effective amount of the steroid or a pharmaceutically acceptable salt thereof administered to a CAH patient without receiving a CRF1 antagonist (i.e., a compound disclosed herein) or a pharmaceutically acceptable salt thereof. In some embodiments, the subject has an ACTH level that is more than 1.75 times the upper limit of the normal range of ACTH and an A4 level that is more than 1.5 times the upper limit of the normal range of A4. In some embodiments, the subject has an ACTH level that is more than 1.5 times the upper limit of the normal range of ACTH and an A4 level that is more than 1.25 times the upper limit of the normal range of A4. In some embodiments, the subject has an ACTH level that is more than 1.25 times the upper limit of the normal range of ACTH and an A4 level that is more than 1 times the upper limit of the normal range of A4. In some embodiments, the subject has an ACTH level that is more than 1 times the upper limit of the normal range of ACTH and an A4 level that is more than 1.25 times the upper limit of the normal range of A4. In some embodiments, the subject has an ACTH level that is more than 1.25 times the upper limit of the normal range of ACTH and an A4 level that is more than 1.5 times the upper limit of the normal range of A4. In some embodiments, the subject has an ACTH level that is more than 1.5 times the upper limit of the normal range of ACTH and an A4 level that is more than 1.75 times the upper limit of the normal range of A4.

In some embodiments, a dose of about 10 mg to about 80 mg GC is administered to a CAH patient when a CRF1 antagonist is not administered. In some embodiments, the dose of GC is administered to CAH daily one time, two times, three times, or four times. In some embodiments, a corticotropin-releasing factor type-1 (CRF1) antagonist or a pharmaceutically acceptable salt thereof in a dose of about 10 mg to about 200 mg total daily dose is administered to the subject. For example, not meant to be limiting, a dose of about 80 mg GC administered to a CAH patient when a CRF1 antagonist is not administered. If this CAH patient's ACTH level or A4 level is more than the upper limit of the normal range of ACTH level or A4 level after controlled by factors such as age, sex, menopausal status, laboratory setting, the dose of 80 mg GC may be maintained when administered together the CRF1 antagonist (i.e., a compound disclosed herein). Further, if this CAH patient's ACTH level and A4 levels are more than the upper limit of the normal range of ACTH level and A4 level respectively after controlled by factors such as age, sex, menopausal status, laboratory setting, the dose of 80 mg GC may be maintained when administered together the CRF1 antagonist (i.e., a compound disclosed herein).

During the treatment of administering the maintained dose of GC together with the CRF1 antagonist, the CAH patient's ACTH and A4 levels are monitored. In some embodiments, as soon as the CAH patient's ACTH and A4 level become less than 2 times the upper limit of the normal range of the ACTH and A4 level respectively, the dose of GC may be reduced in a 5 mg hydrocortisone equivalent increment to the CAH patient when administered together with the CRF1 antagonist. In some embodiments, as soon as the CAH patient's ACTH and A4 levels become less than 1.75 times the upper limit of the normal range of the ACTH and A4 level respectively, the dose of GC may be reduced in a 5 mg hydrocortisone equivalent increment to the CAH patient when administered together with the CRF1 antagonist. In some embodiments, as soon as the CAH patient's ACTH and A4 levels become less than 1.5 times the upper limit of the normal range of the ACTH and A4 level respectively, the dose of GC may be reduced in a 5 mg hydrocortisone equivalent increment to the CAH patient when administered together with the CRF1 antagonist. In some embodiments, as soon as the CAH patient's ACTH and A4 levels become less than 1.25 times the upper limit of the normal range of the ACTH and A4 levels respectively, the dose of GC may be reduced in a 5 mg hydrocortisone equivalent increment to the CAH patient when administered together with the CRF1 antagonist. In some embodiments, as soon as the CAH patient's ACTH and A4 levels become less than 1.0 times the upper limit of the normal range of the ACTH and A4 levels respectively, the dose of GC may be reduced in a 5 mg hydrocortisone equivalent increment to the CAH patient when administered together with the CRF1 antagonist. Moreover, the dose of the GC may be continuously reduced in a 5 mg increment when administered together with the CRF1 antagonist until the subject's ACTH and A4 levels are not less than 2, 1.5, or 1.25 times the upper limit of the normal range of ACTH and A4 levels respectively.

In some embodiments, the subject will be treated for a period of about 1 week to about 40 weeks. In some embodiments, the subject will be treated for a period of about 2 weeks to about 39 weeks. In some embodiments, the subject will be treated for a period of about 3 weeks to about 38 weeks. In some embodiments, the subject will be treated for a period of about 4 weeks to about 36 weeks. In some embodiments, after the subject has been treated for a period of time described herein, the subject may be reassessed to determine whether a different patient subtype regimen may be applicable.

In some embodiments, the dose of steroid or a pharmaceutically acceptable salt thereof is in a dose of about 10 mg to about 80 mg of hydrocortisone equivalents. In some embodiments, the steroid is a glucocorticoid or a pharmaceutically acceptable salt thereof. In some embodiments, the subject is tested in the morning to determine morning levels of A4 and ACTH.

With specificity provided from a particular laboratory setting, in some embodiments, two times the upper limit of the normal laboratory reference range of A4 for a male subject is more than about 440 ng/dl, more than about 430 ng/dl, more than about 420 ng/dl, more than about 410 ng/dl, more than about 400 ng/dl, more than about 390 ng/dl, more than about 380 ng/dl, more than about 370 ng/dl. In some embodiments, the normal laboratory reference range of A4 for a male subject is from about 40 ng/dl to about 190 ng/dl. In some embodiments, the normal laboratory reference range of A4 for a male subject is from about 50 ng/dl to about 220 ng/dl.

In some embodiments, two times the upper limit of the normal laboratory reference range of A4 for a female subject is more than about 560 ng/dl, more than about 550 ng/dl, more than about 540 ng/dl, more than about 530 ng/dl, more than about 520 ng/dl, more than about 510 ng/dl, 500 ng/dl, more than about 490 ng/dl, more than about 480 ng/dl, more than about 470 ng/dl, more than about 460 ng/dl, more than about 450 ng/dl, more than about 430 ng/dl, more than about 420 ng/dl, more than about 410 ng/dl, 400 ng/dl, more than about 390 ng/dl, more than about 380 ng/dl, more than about 370 ng/dl, more than about 360 ng/dl, more than about 350 ng/dl, or more than about 340 ng/dl. In some embodiments, the normal laboratory reference range of A4 for a female subject is from about 51 ng/dl to about 213 ng/dl. In some embodiments, the normal laboratory reference range of A4 for a female subject is from about 73 ng/dl to about 230 ng/dl. In some embodiments, the normal laboratory reference range of A4 for a female subject is from about 73 ng/dl to about 184 ng/dl.

In some embodiments, two time the upper limit of the normal laboratory reference of ACTH is more than about 110 pg/dl, more than about 120 pg/dl, more than about 130 pg/dl, more than about 140 pg/dl, or more than about 150 pg/dl. In some embodiments, the normal laboratory reference range of ACTH is from about 7 pg/dl to about 70 pg/dl.

In some embodiments, the CRF1 antagonist or pharmaceutically acceptable salt thereof is selected from the group consisting of: Antalarmin hydrochloride, Pfizer CP 154526, CP 376395 hydrochloride, NBI 27914 hydrochloride, NBI 35965 hydrochloride, NGD 98-2 hydrochloride, Pexacerfont, R 121919 hydrochloride, SNO03.

In some embodiments, the CRF1 antagonist is a compound of Formula (I):

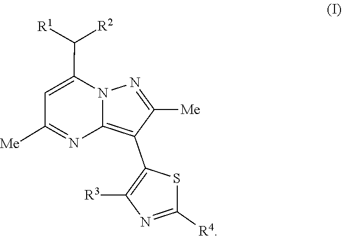

or a pharmaceutically acceptable salt thereof, wherein:
R¹ and R² are independently ethyl or n-propyl;
R³ is hydrogen, Cl, Br, methyl, trifluoromethyl, or methoxy; and
R⁴ is hydrogen, Br, R^{a}R^{b}N—, methoxymethyl, n-butyl, acetamido, pyridin-4-yl, morpholin-4-yl,

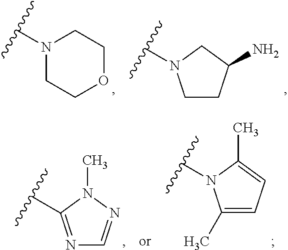

and
R^{a} and R^{b} are independently hydrogen, C₁-C₃alkyl, H₂NCH₂CH₂—, (CH₃)₃COC(O)NHCH₂CH₂—, or CH₃CH₂CH₂NHCH₂CH₂—.

In some embodiments, R³ is Cl, Br, methyl, or trifluoromethyl. In some embodiments, R³ is Cl, Br, or methyl.

In some embodiments, R⁴ is Br, R^{a}R^{b}N—, pyridin-4-yl, morpholin-4-yl, or

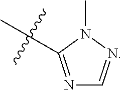 [SMILES: *c1ncnn1C]

In some embodiments, R⁴ is morpholin-4-yl or

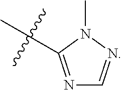 [SMILES: *c1ncnn1C]

In some embodiments, R⁴ is hydrogen, Br, R^{a}R^{b}N— and R^{a} and R^{b} are independently C₁-C₃alkyl.

In some embodiments, the compound is

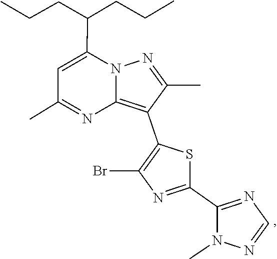 [SMILES: CCCC(CCC)c1cc(C)nc2c(-c3sc(-c4ncnn4C)nc3Br)c(C)nn12]

or a pharmaceutically acceptable salt thereof.

In some embodiments, the compound is

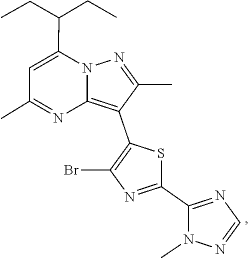 [SMILES: CCC(CC)c1cc(C)nc2c(-c3sc(-c4ncnn4C)nc3Br)c(C)nn12]

or a pharmaceutically acceptable salt thereof.

In some embodiments, the compound is

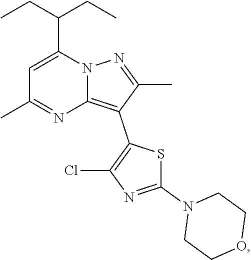 [SMILES: CCC(CC)c1cc(C)nc2c(-c3sc(N4CCOCC4)nc3Cl)c(C)nn12]

or a pharmaceutically acceptable salt thereof.

In some embodiments, the CRF1 antagonist or pharmaceutically acceptable salt is administered in a dose of about 25 mg to about 200 mg total daily dose to the subject. In some embodiments, the CRF1 antagonist or pharmaceutically acceptable salt is administered in a dose of about 200 mg total daily dose to the subject.

In some embodiments, the CRF1 antagonist or pharmaceutically acceptable salt is administered in a dose of about 150 mg total daily dose to the subject. In some embodiments, the CRF1 antagonist or pharmaceutically acceptable salt is administered in a dose of about 100 mg total daily dose to the subject. In some embodiments, the CRF1 antagonist or pharmaceutically acceptable salt is administered in a dose of about 50 mg total daily dose to the subject. In some embodiments, the CRF1 antagonist or pharmaceutically acceptable salt is administered in a dose of about 40 mg total daily dose to the subject. In some embodiments, the CRF1 antagonist or pharmaceutically acceptable salt is administered in a dose of about 30 mg total daily dose to the subject. In some embodiments, the CRF1 antagonist or pharmaceutically acceptable salt is administered in a dose of about 25 mg total daily dose to the subject. In some embodiments, the CRF1 antagonist or pharmaceutically acceptable salt is administered in a dose of about 20 mg total daily dose to the subject. In some embodiments, the CRF1 antagonist or pharmaceutically acceptable salt is administered in a dose of about 15 mg total daily dose to the subject. In some embodiments, the CRF1 antagonist or pharmaceutically acceptable salt is administered in a dose of about 10 mg total daily dose to the subject. In some embodiments, the CRF1 antagonist or pharmaceutically acceptable salt is administered in a dose of about 5 mg total daily dose to the subject.

In some embodiments, the CRF1 antagonist or pharmaceutically acceptable salt is in the form of microparticles. In some embodiments, the average size of the microparticles is between about 1 μm to about 20 μm. In some embodiments, the average size of the microparticles is between about 5 μm to about 15 μm. In some embodiments, the average size of the microparticles is less than about 10μ.

In some embodiments, the CRF1 antagonist or pharmaceutically acceptable salt thereof is administered as a pharmaceutical composition. In some embodiments, the steroid or a pharmaceutically acceptable salt thereof is administered as a pharmaceutical composition.

In some embodiments, the pharmaceutical composition is in the form of a capsule or a tablet. In some embodiments, the capsule is a hard gelatin capsule. In some embodiments, the capsule is a soft gelatin capsule. In some embodiments, the capsule is formed using materials selected from the group consisting of natural gelatin, synthetic gelatin, pectin, casein, collagen, protein, modified starch, polyvinylpyrrolidone, acrylic polymers, cellulose derivatives, and any combinations thereof.

In some embodiments, the pharmaceutical composition is free of additional excipients. In some embodiments, the pharmaceutical composition further comprises one or more pharmaceutically acceptable excipients. In some embodiments, the pharmaceutical composition is in the form of a tablet. In some embodiments, the pharmaceutical composition further comprises one or more pharmaceutically acceptable excipients.

In some embodiments, CRF1 antagonist or pharmaceutically acceptable salt is formulated as a capsule or a tablet as to provide a Tmax of about 1 to about 8 hours in a subject. In some embodiments, CRF1 antagonist or pharmaceutically acceptable salt is formulated as a capsule or a tablet as to provide a Tmax of about 2 to about 7 hours in a subject. In some embodiments, CRF1 antagonist or pharmaceutically acceptable salt is formulated as a capsule or a tablet as to provide a Tmax of about 2 to about 6 hours in a subject. In some embodiments, CRF1 antagonist or pharmaceutically acceptable salt is formulated as a capsule or a tablet as to provide a Tmax of about 3 to about 5 hours in a subject.

In some embodiments, CRF1 antagonist or pharmaceutically acceptable salt is formulated as a capsule or a tablet as to provide a Tmax of about 8 hours in a subject. In some embodiments, CRF1 antagonist or pharmaceutically acceptable salt is formulated as a capsule or a tablet as to provide a Tmax of about 7 hours in a subject. In some embodiments, CRF1 antagonist or pharmaceutically acceptable salt is formulated as a capsule or a tablet as to provide a Tmax of about 6 hours in a subject. In some embodiments, CRF1 antagonist or pharmaceutically acceptable salt is formulated as a capsule or a tablet as to provide a Tmax of about 5 hours in a subject. In some embodiments, CRF1 antagonist or pharmaceutically acceptable salt is formulated as a capsule or a tablet as to provide a Tmax of about 4 hours in a subject. In some embodiments, CRF1 antagonist or pharmaceutically acceptable salt is formulated as a capsule or a tablet as to provide a Tmax of about 3 hours in a subject. In some embodiments, CRF1 antagonist or pharmaceutically acceptable salt is formulated as a capsule or a tablet as to provide a Tmax of about 2 hours in a subject. In some embodiments, CRF1 antagonist or pharmaceutically acceptable salt is formulated as a capsule or a tablet as to provide a Tmax of about 1 hour in a subject.

In some embodiments, the methods described herein include administration of the pharmaceutical composition comprising CRF1 antagonist or pharmaceutically acceptable salt, or a pharmaceutically acceptable salt or solvate thereof once a month, twice a month, three times a month, once a week, twice a week, three times a week, once every two days, once a day, twice a day, three times a day, or four times a day. In some embodiments, the methods described herein administer CRF1 antagonist or pharmaceutically acceptable salt, or a pharmaceutically acceptable salt or solvate thereof once a day. In some embodiments, the methods described herein administer CRF1 antagonist or pharmaceutically acceptable salt, or a pharmaceutically acceptable salt or solvate thereof twice a day.

In some embodiments, the methods described herein include administration of about 1 mg to about 2000 mg of CRF1 antagonist or pharmaceutically acceptable salt, or a pharmaceutically acceptable salt or solvate thereof, per day. In some embodiments, CRF1 antagonist or pharmaceutically acceptable salt is administered at a dose between about 50 mg/day and about 1600 mg/day. In some embodiments, Compound 1 is administered at a dose between about 50 mg/day and about 1500 mg/day. In some embodiments, Compound 1 is administered at a dose between about 50 mg/day and about 1400 mg/day. In some embodiments, Compound 1 is administered at a dose between about 50 mg/day and about 1300 mg/day. In some embodiments, Compound 1 is administered at a dose between about 50 mg/day and about 1200 mg/day. In some embodiments, Compound 1 is administered at a dose between about 50 mg/day and about 1100 mg/day. In some embodiments, Compound 1 is administered at a dose between about 50 mg/day and about 1000 mg/day. In some embodiments, Compound 1 is administered at a dose between about 50 mg/day and about 900 mg/day. In some embodiments, Compound 1 is administered at a dose between about 50 mg/day and about 800 mg/day. In some embodiments, Compound 1 is administered at a dose between about 60 mg/day and about 800 mg/day. In some embodiments, Compound 1 is administered at a dose between about 70 mg/day and about 800 mg/day. In some embodiments, Compound 1 is administered at a dose between about 80 mg/day and about 800 mg/day. In some embodiments, Compound 1 is administered at a dose between about 90 mg/day and about 800 mg/day. In some embodiments, Compound 1 is administered at a dose between about 100 mg/day and about 800 mg/day. In some embodiments, Compound 1 is administered at a dose between about 100 mg/day and about 700 mg/day. In some embodiments, Compound 1 is administered at a dose between about 100 mg/day and about 600 mg/day. In some embodiments, Compound 1 is administered at a dose between 150 mg/day and about 600 mg/day. In some embodiments, Compound 1 is administered at a dose between 200 mg/day and about 600 mg/day. In some embodiments, Compound 1 is administered at a dose between 200 mg/day and about 500 mg/day. In some embodiments, Compound 1 is administered at a dose between 200 mg/day and about 400 mg/day.

In some embodiments, Compound 1 is administered at a dose of about 500 mg/day. In some embodiments, Compound 1 is administered at a dose of about 400 mg/day. In some embodiments, Compound 1 is administered at a dose of about 300 mg/day. In some embodiments, Compound 1 is administered at a dose of about 200 mg/day. In some embodiments, Compound 1 is administered at a dose of about 100 mg/day.

In some embodiments, about 100 mg to about 1600 mg of CRF1 antagonist or pharmaceutically acceptable salt, or a pharmaceutically acceptable salt or solvate thereof, is administered per day. In some embodiments, about 200 mg to about 1600 mg of CRF1 antagonist or pharmaceutically acceptable salt, or a pharmaceutically acceptable salt or solvate thereof, is administered per day. In some embodiments, about 200 mg to about 1200 mg of CRF1 antagonist or pharmaceutically acceptable salt, or a pharmaceutically acceptable salt or solvate thereof, is administered per day. In some embodiments, about 200 mg to about 1000 mg of CRF1 antagonist or pharmaceutically acceptable salt, or a pharmaceutically acceptable salt or solvate thereof, is administered per day. In some embodiments, about 200 mg to about 800 mg of CRF1 antagonist or pharmaceutically acceptable salt, or a pharmaceutically acceptable salt or solvate thereof, is administered per day. In some embodiments, about 100 mg to about 800 mg of CRF1 antagonist or pharmaceutically acceptable salt, or a pharmaceutically acceptable salt or solvate thereof, is administered per day. In some embodiments, about 200 mg to about 800 mg of CRF1 antagonist or pharmaceutically acceptable salt, or a pharmaceutically acceptable salt or solvate thereof, is administered per day. In some embodiments, about 100 mg to about 600 mg of CRF1 antagonist or pharmaceutically acceptable salt, or a pharmaceutically acceptable salt or solvate thereof, is administered per day. In some embodiments, about 200 mg to about 600 mg of CRF1 antagonist or pharmaceutically acceptable salt is administered per day. In some embodiments, about 300 mg to about 600 mg of CRF1 antagonist or pharmaceutically acceptable salt, or a pharmaceutically acceptable salt or solvate thereof, is administered per day. In some embodiments, about 100 mg to about 400 mg of CRF1 antagonist or pharmaceutically acceptable salt, or a pharmaceutically acceptable salt or solvate thereof, is administered per day. In some embodiments, about 200 mg to about 400 mg of CRF1 antagonist or pharmaceutically acceptable salt, or a pharmaceutically acceptable salt or solvate thereof, is administered per day. In some embodiments, about 300 mg to about 400 mg of CRF1 antagonist or pharmaceutically acceptable salt, or a pharmaceutically acceptable salt or solvate thereof, is administered each day.

In some embodiments, less than about 2000 mg CRF1 antagonist or pharmaceutically acceptable salt, or a pharmaceutically acceptable salt or solvate thereof, is administered per day. In some embodiments, less than about 1800 mg CRF1 antagonist or pharmaceutically acceptable salt, or a pharmaceutically acceptable salt or solvate thereof, is administered per day. In some embodiments, less than about 1600 mg CRF1 antagonist or pharmaceutically acceptable salt, or a pharmaceutically acceptable salt or solvate thereof, is administered per day. In some embodiments, less than about 1400 mg CRF1 antagonist or pharmaceutically acceptable salt, or a pharmaceutically acceptable salt or solvate thereof, is administered per day. In some embodiments, less than about 1200 mg CRF1 antagonist or pharmaceutically acceptable salt, or a pharmaceutically acceptable salt or solvate thereof, is administered per day. In some embodiments, less than about 1000 mg CRF1 antagonist or pharmaceutically acceptable salt, or a pharmaceutically acceptable salt or solvate thereof, is administered per day. In some embodiments, less than about 800 mg CRF1 antagonist or pharmaceutically acceptable salt, or a pharmaceutically acceptable salt or solvate thereof, is administered per day. In some embodiments, less than about 600 mg CRF1 antagonist or pharmaceutically acceptable salt, or a pharmaceutically acceptable salt or solvate thereof, is administered per day. In some embodiments, less than about 500 mg CRF1 antagonist or pharmaceutically acceptable salt, or a pharmaceutically acceptable salt or solvate thereof, is administered per day. In some embodiments, less than about 400 mg CRF1 antagonist or pharmaceutically acceptable salt, or a pharmaceutically acceptable salt or solvate thereof, is administered per day. In some embodiments, less than about 300 mg CRF1 antagonist or pharmaceutically acceptable salt, or a pharmaceutically acceptable salt or solvate thereof, is administered per day. In some embodiments, less than about 200 mg CRF1 antagonist or pharmaceutically acceptable salt, or a pharmaceutically acceptable salt or solvate thereof, is administered per day.

In some embodiments, the methods described herein include administration of the pharmaceutical compositions described herein wherein the subject is in the fed state. In some embodiments, the methods described herein include administration of the pharmaceutical compositions described herein wherein the subject is in the fasted state.

In some embodiments, the methods described herein include administration of the pharmaceutical compositions described herein at bedtime.

In some embodiments, the methods described herein include administration of the pharmaceutical compositions described herein less than about 4 hours before sleep. In some embodiments, the methods described herein include administration of the pharmaceutical compositions described herein less than about 3 hours before sleep. In some embodiments, the methods described herein include administration of the pharmaceutical compositions described herein less than about 2 hours before sleep. In some embodiments, the methods described herein include administration of the pharmaceutical compositions described herein less than about 1 hour before sleep. In some embodiments, the methods described herein include administration of the pharmaceutical compositions described herein less than about 30 mins before sleep.

In some embodiments, the methods described herein include administration of the pharmaceutical compositions described herein in the evening.

In some embodiments, the methods described herein include administration of the pharmaceutical compositions described herein at about 11 pm at night. In some embodiments, the methods described herein include administration of the pharmaceutical compositions described herein at about 10 pm at night. In some embodiments, the methods described herein include administration of the pharmaceutical compositions described herein at about 9 pm at night. In some embodiments, the methods described herein include administration of the pharmaceutical compositions described herein at about 8 pm at night.

In some embodiments, the steroid or pharmaceutically acceptable salt thereof is a glucocorticoid or a pharmaceutically acceptable salt thereof. In some embodiments, the glucocorticoid or a pharmaceutically acceptable salt there is prednisone, cortisone, prednisolone, triamcinolone, methylprednisolone, betamethasone, dexamethasone, hydrocortisone or a pharmaceutically acceptable salt thereof.

In some embodiments, the levels of A4 and ACTH in the subject are determined from a biological sample from the subject. In some embodiments, the biological sample is selected from the group of blood, blood fractions, plasma, serum, urine, other types of bodily secretions, and saliva. In some embodiments, the biological sample is obtained non-invasively.

In some embodiments, the subject is a pediatric patient. In some embodiments, the subject is from about 0 years old to about 18 years old. In some embodiments, the subject is an adult patient.

In some embodiments, the steroid and the CRF1 antagonist are administered concurrently. In some embodiments, the steroid and the CRF1 antagonist are administered in one pharmaceutical composition. In some embodiments, the steroid and the CRF1 antagonist are administered concurrently in separate pharmaceutical compositions. In some embodiments, the steroid and the CRF1 antagonist are administered sequentially.

In some embodiments, the steroid and the CRF1 antagonist are administered sequentially within 24 hours. In some embodiments, the steroid and the CRF1 antagonist are administered sequentially within 8 hours. In some embodiments, the steroid and the CRF1 antagonist are administered sequentially within 2 hours. In some embodiments, the steroid and the CRF1 antagonist are administered sequentially within 10 minutes.

In some embodiments, CAH is classic CAH. In some embodiments, CAH is non-classic CAH.

In some embodiments, the glucocorticoid is beclomethasone, betamethasone, budesonide, cortisone, dexamethasone, hydrocortisone, methylprednisolone, prednisolone, prednisone, or triamcinolone. In some embodiments, the glucocorticoid is hydrocortisone.

In some embodiments, the glucocorticoid is hydrocortisone and the dose administered is less than the recommended dose of 15-25 mg/day.

In some embodiments, the glucocorticoid is prednisone and the dose administered is less than the recommended dose of 5-7.5 mg/day.

In some embodiments, the glucocorticoid is prednisolone and the dose administered is less than the recommended dose of 4-6 mg/day.

In some embodiments, the glucocorticoid is dexamethasone and the dose administered is less than the recommended dose of 0.25-0.5 mg/day.

### EXAMPLES

The following examples further illustrate the invention but should not be construed as in any way limiting its scope. In particular, the processing conditions are merely exemplary and can be readily varied by one of ordinary skill in the art.

All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. Unless defined otherwise, technical and scientific terms used herein have the same meaning as is commonly understood by one of skill in the art to which this invention belongs.

### Example 1—Identifying Discrete Subgroups of Patients with CAH Based on A4 and ACTH Levels in a Subject Who has CAH

Study Compound 3, a Phase 2, open-label, first-in-CAH-patients, proof-of-concept, dose ranging study, evaluated the safety and efficacy of repeated doses of SPR001 in adults with classic CAH. The population (n=24; with 2 subjects completing 2 cohorts) consisted of subjects with classic CAH that is not adequately controlled despite a stable glucocorticoid regimen based on the assessment of a single hormone at study screening, 17-OHP >800 ng/dl, which is 4 times the upper limit of a normal reference range of 17-OHP level. 17-OHP was chosen as the hormone as an inclusion criterion, as at the time, it represented the state-of-art in CAH for assessment of disease severity and adequacy of glucocorticoid therapy. The study comprised 3 cohorts, Cohort A was a 6-week intra-subject dose escalation treatment period with three 2-week treatment periods of 200 mg glucocorticoid (QD) (only daily), 600 mg QD and 1000 mg QD without washout between treatment periods. Cohorts B and C consisted of 2-week treatment periods of either 100 bis in die (BID) (twice daily) (Cohort C) or 200 mg BID (Cohort B). At baseline, the following hormones were assessed, 17-OHP, ACTH and A4. The same hormones were assessed at the end of each 2-week treatment period.

The inclusion criterion of an elevated 17-OHP (>800 ng/dL) was expected, based on the current state-of-art, to enroll subjects with elevated ACTH, a precursor to 17-OHP, and elevated A4, a downstream androgen of 17-OHP. An elevated 17-OHP corresponded to both elevated and non-elevated pre (ACTH) and post (A4) stream hormones. This is evidenced in Table 1. All cohorts met the criteria of elevated 17-OHP as evidenced in the geometric means. Subjects in Cohort B, while having elevated 17-OHP, did not have elevated ACTH or elevated A4. Cohorts A and C had all 3 hormones elevated corresponding to the state-of-the-art.

TABLE 1
Cohorts assessed in a Phase 2, open-label, first-in-CAH-patients,
proof-of-concept, dose ranging study

| | Cohort A | Cohort B | Cohort C |
|---|---|---|---|
| | Compound 3 | Compound 3 | Compound 3 |
| | 200/600/1000 QD | 200 BID | 100 BID |
| | (N = 10) | (N = 8) | (N = 7) |
| Screening 17-OHP, ng/dL | | | |
| Geometric Mean (CV %) | 3887.3 (125.6) | 3157.8 (109.1) | 6106.8 (113.6) |
| Baseline (8am) 17-OHP, ng/dL | | | |
| Geometric Mean (CV %) | 7362.0 (95.2) | 1954.2 (344.2) | 6753.4 (96.0) |
| Baseline (8 am) ACTH, pg/mL | | | |
| Geometric Mean (CV %) | 306.72 (97.1) | 27.17 (190.6) | 292.45 (100.9) |
| Baseline (8am) A4, ng/dL | | | |
| Geometric Mean (CV %) | 286.9 (168.1) | 93.0 (194.6) | 242.2 (121.8) |

A clustering approach, supported and guided by clinical input, was used to define subgroups based on baseline hormone control of A4 and ACTH. The inclusion of 17-OHP as an input occluded the identification of clear subgroups which was non-obvious.

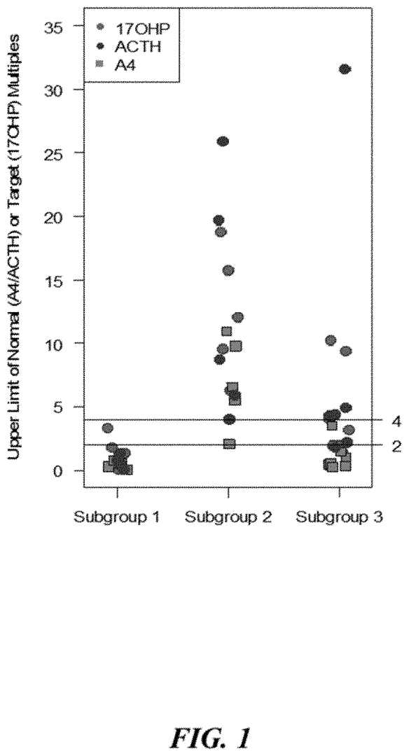

Three subgroups of patients were identified (see FIG. 1):
Subgroup One: Subjects with A4 and ACTH levels generally lower than two-fold the upper limit of normal for each laboratory assessment respectively.
Subgroup Two: Subjects with A4 and ACTH generally higher than two-fold the upper limit of normal for each laboratory assessment respectively.
Subgroup Three: Subjects with either A4 or ACTH above two fold and the other below two-fold the upper limit of normal for each laboratory assessment respectively.
Subgroups 2 and 3 can be combined into a single subgroup.

By classifying the patients into subgroup types based on A4 and ACTH, it provides an alternative method to assessing diseases severity of subjects based on 17-OHP and A4. In addition, these subgroups provide a basis for different, yet sequential, methods of treatment.

### Example 2—Phase 2 Clinical Studies

A clinical study is conducted involving up to 52 weeks treatment of Compound 3 with concurrent daily glucocorticoid therapy for the treatment of adults with classic CAH. After screening using A4 and ACTH level criteria, eligible patients are enrolled into up to 12 weeks of placebo-controlled treatment followed by 40 weeks of open label treatment and a 4-week washout/safety follow-up period.

The population are composed of subjects from subgroup 2, approximately n=18 per treatment group and a range of doses between 50 mg to 200 mg and placebo. Each subject receives Compound 3 or placebo and a glucocorticoid daily for up to 12 weeks in a double-blind matter. Following the placebo-controlled period, all subjects receive open label compound 3 as various doses and a glucocorticoid will be administered as an oral daily dose as in the double-blind period. Patients self-administer Compound 3 for up to 52 weeks. The maximum treatment period for placebo is 12 weeks.

Study Design
Study Type: Interventional
Primary Purpose: Treatment
Study Phase: Phase 2
Interventional Study Model: double-blind, placebo controlled (12 weeks), open label (up to 40 weeks)
Allocation: Equal Randomization
Enrollment: Up to approximately 72

### Outcome Measures

### Primary Outcome Measures:

1. Assess reduction in key hormones such as ACTH, A4 and 17-OHP.

### Secondary Outcome Measures:

2. Assess normalization proportions for key hormones such as ACTH, A4 and 17-OHP.

Eligibility
Minimum Age: 16 Years
Maximum Age: 65 Years
Sex: All
Gender Based: No
Accepts Healthy Volunteers: No
Criteria: Inclusion

### Criteria:

### Inclusion Criteria:

Male and female patients age 18 or older.
Documented diagnosis of classic CAH due to 21-hydroxylase deficiency
Elevated A4 and ACTH more than two times the upper limit of normal laboratory reference range at screening and baseline
On a stable glucocorticoid replacement regimen for a minimum of 30 days

### Exclusion Criteria:

Clinically significant unstable medical condition, illness, or chronic disease
Clinically significant psychiatric disorder.
Clinically significant abnormal laboratory finding or assessment
History of bilateral adrenalectomy or hypopituitarism
Pregnant or nursing females
Use of any other investigational drug within 30 days
Unable to understand and comply with the study procedures, understand the risks, and/or unwilling to provide written informed consent.

The disclosed compound 3 is an inhibitor of the CYP3A4 pathway and dexamethasone (DEX) is primarily metabolized through the CYP3A4 pathway. As part of the study, pre and pose concentrations of DEX and cortisol were measured. In some embodiments, the biomarker reduction analysis comprises all subjects. In some embodiments, the biomarker reduction analysis does not comprise DEX-treated subjects.

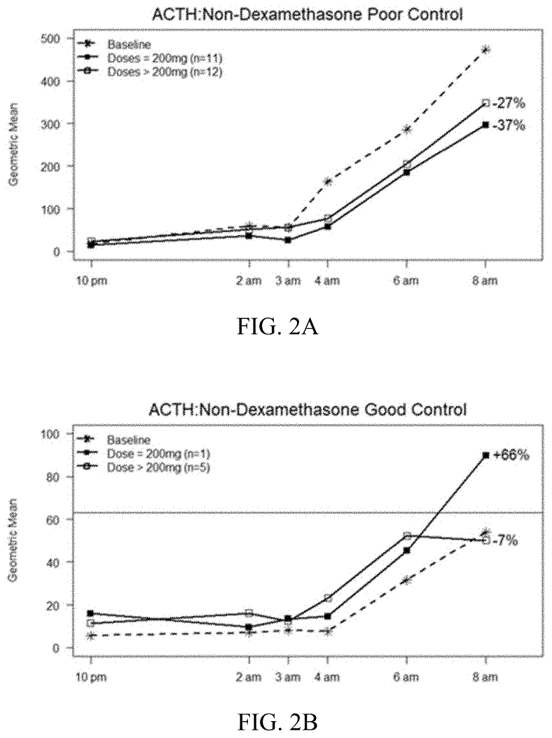

As shown in FIGS. 2A and 2B, time matched changes among different treatment groups of ACTH level measured from 10 pm the day before to 8 am the next day are illustrated in non-dexamethasone subjects. A pooled baseline (dotted) and 2 lines (solid) representing two dose groups (doses=200 mg and doses >200 mg) using the percentage change from dose group baseline presented as percentage change from the pooled baseline. The figures are divided by disease control group, poor disease control with highly elevated ACTH and A4 and the good disease control group with ACTH and A4 that is near normal or below normal ranges. Each group has elevated 17-OHP.

According to FIG. 2A, in the poor disease group the geometric means post 4 am are highly elevated reaching values greater than 400 pg/mL or greater than 6× upper limit of normal (ULN)(63.3 pg/mL). Treatment with compound 3 reduced the magnitude of the increase in ACTH post 3 am demonstrating an improvement in the serial hormone profile. According to FIG. 2B, in the good disease control group, the baseline serial profile was below the ULN throughout the evaluated time period. Treatment with compound 3 generally maintained the serial profile below the ULN. The observed changes are likely representative of intra-subject daily variation.

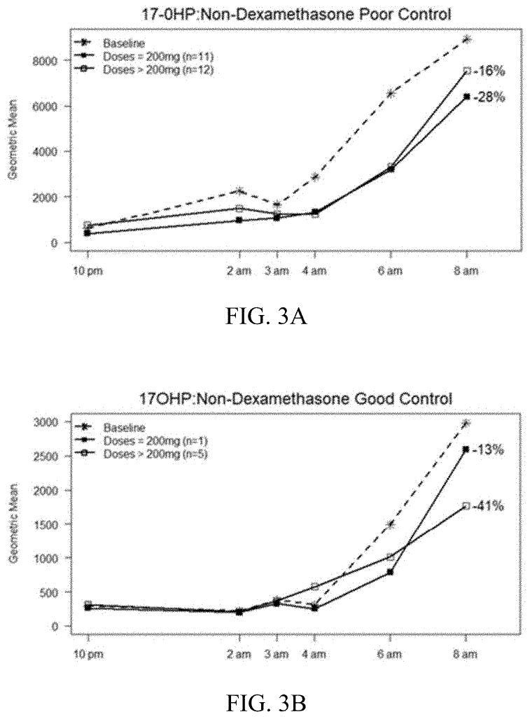

As shown in FIGS. 3A and 3B, the time matched changes among different treatment groups of 17-OHP level measured from 10 pm the day before to 8 am the next day are illustrated in non-dexamethasone subjects. A pooled baseline (dotted) and 2 lines (solid) representing two dose groups (doses=200 mg and doses >200 mg) using the percentage change from dose group baseline presented as percentage change from the pooled baseline. The figures are divided by disease control group, poor disease control with highly elevated ACTH and A4 and the good disease control group with ACTH and A4 that is near normal or below normal ranges. In both groups, 17-OHP is elevated above its target (1200 ng/dL). The Y-axis is different with the poor disease control group exceeding 8000 ng/dL in FIG. 3A and the good disease group was near an upper bound of approximately 3000 ng/dL in FIG. 3B. In both groups, treatment with compound 3 reduced the magnitude of the increase in 17-OHP post 3 am demonstrating an improvement in the serial hormone profile.

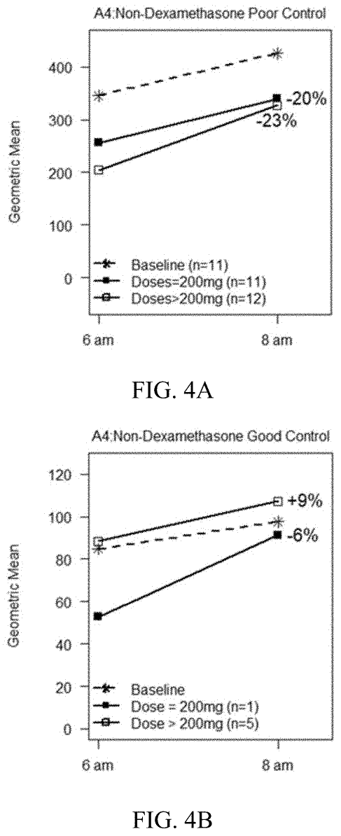

As shown in FIGS. 4A and 4B, the time matched changes among different treatment groups of A4 level measured from 6 am to 8 am the same day in non-dexamethasone subjects. A pooled baseline (dotted) and 2 lines (solid) representing two dose groups (doses=200 mg and doses >200 mg) using the percentage change from dose group baseline presented as percentage change from the pooled baseline. The figures are divided by disease control group, poor disease control with highly elevated ACTH and A4 and the good disease control group with ACTH and A4 that is near normal or below normal ranges. Each group has elevated 17-OHP.

As shown in FIG. 4A, the baseline profile for the poor disease control group is above the blended gender based ULN of approximately 212 ng/dL. Treatment with compound 3 improves the A4 profile in subjects toward the blended gender based ULN of approximately 212 ng/dL. As shown in FIG. 4B, the baseline profile for the good disease control group is well below the blended gender based ULN of approximately 212 ng/dL. Treatment with compound 3 was able to further reduce A4 level compared to the baseline level. The small variation about the baseline likely representative of intra-subject daily variation.

### Example 3—Phase 2 Clinical Studies

A Phase 2 Study is conducted involving a up to 52 weeks treatment multiple-dose study of Compound 3 and a glucocorticoid for the treatment of adults with classic CAH. After screening with the criteria disclosed herein, eligible patients are enrolled into up to 52 weeks of treatment followed by a 4-week washout/safety follow-up period.

The population are composed of subgroup one subjects, approximately n=30 with a dose of 200 mg and n=30 with placebo. Each subject receives Compound 3 or placebo and a glucocorticoid daily for up to 24 weeks in a double-blind manner. Compound 3 and a glucocorticoid are administered as an oral daily dose. Following the double-blind period, all subject will receive open label compound 3 at 200 mg. Patients self-administer Compound 3 for up to 52 weeks. After a minimum of 2 weeks of concomitant treatment of Compound 3 and a glucocorticoid, subjects are eligible for reductions in their glucocorticoid. Reduction occurs at 5 mg hydrocortisone equivalent increments over a period of 24 weeks with subjects eligible for up to 5 dose reductions prior to study end.

Study Design
Study Type: Interventional
Primary Purpose: Treatment
Study Phase: Phase 2
Interventional Study Model: double-blind (24 weeks) open-label (28 weeks)
Allocation: Equal Randomization
Enrollment: Up to approximately 60 [n=30 per group]

### Outcome Measures

### Primary Outcome Measure:

1. Reduction of glucocorticoid dose

### Secondary Outcome Measure:

2. Assess normalization proportions for each key hormone
3. Assess impact of reductions of glucocorticoid dose on signs and symptoms of CAH
4. Assess impact of reductions of glucocorticoid dose on overall glucocorticoid toxicity Index.

### Eligibility

Minimum Age: 18 Years
Maximum Age: 65
Sex: All
Gender Based: No
Accepts Healthy Volunteers: No
Criteria: Inclusion

### Criteria:

### Inclusion Criteria:

Male and female patients age 18 or older.
Documented diagnosis of classic CAH due to 21-hydroxylase deficiency
Elevated A4 and ACTH <1.5× upper limit of normal at screening and baseline
On a stable glucocorticoid replacement regimen for a minimum of 30 days

### Exclusion Criteria:

Clinically significant unstable medical condition, illness, or chronic disease
Clinically significant psychiatric disorder.
Clinically significant abnormal laboratory finding or assessment
History of bilateral adrenalectomy or hypopituitarism
Pregnant or nursing females
Use of any other investigational drug within 30 days
Unable to understand and comply with the study procedures, understand the risks, and/or unwilling to provide written informed consent.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of treating Congenital Adrenal Hyperplasia (CAH), comprising administering a steroid or a pharmaceutically acceptable salt thereof and a corticotropin-releasing factor type-1 (CRF₁) antagonist or a pharmaceutically acceptable salt thereof to a subject, wherein when said subject has a level of at least one steroid hormone that is less than two times an upper limit of a reference range of said at least one steroid hormone level, a dose of said steroid or a pharmaceutically acceptable salt thereof is reduced compared to a dose of said steroid or a pharmaceutically acceptable salt thereof administered to a CAH patient that does not receive said CRF₁ antagonist or a pharmaceutically acceptable salt thereof.

2. The method of claim 1, wherein said subject has a level of a second and different steroid hormone that is less than two times an upper limit of a reference range of said second and different steroid hormone level.

3. The method of either claim 1 or claim 2, wherein said steroid hormone is selected from the group consisting of adrenocorticotropic hormone (ACTH), androstenedione (A4), 17-hydroxyprogesterone (17-OHP), deoxycorticosterone, 11-deoxycortisol, cortisol, corticosterone, aldosterone, pregnenolone, 17α-hydroxy pregnenolone, progesterone, dehydroepiandrosterone, androstenediol, testosterone, dihydrotestosterone, estrone, estradiol, and estriol.

4. The method of one of claims 1-3, wherein said subject has a level of 17-OHP that is less than two times an upper limit of a reference range of 17-OHP and a level of ACTH that is less than two times an upper limit of a reference range of ACTH.

5. The method of one of claims 1-3, wherein said subject has a level of 17-OHP that is less than two times an upper limit of a reference range of 17-OHP and a level of A4 that is less than two times an upper limit of a reference range of A4.

6. The method of one of claims 1-3, wherein said subject has a level of A4 that is less than two times an upper limit of a reference range of A4 and a level of ACTH that is less than two times an upper limit of a reference range of ACTH.

7. The method of one of claims 1-3, wherein said subject has a level of 17-OHP that is less than 1.75 times an upper limit of a reference range of 17-OHP and a level of ACTH that is less than 1.75 times an upper limit of a reference range of ACTH.

8. The method of one of claims 1-3, wherein said subject has a level of 17-OHP that is less than 1.75 times an upper limit of a reference range of 17-OHP and a level of A4 that is less than 1.75 times an upper limit of a reference range of A4.

9. The method of one of claims 1-3, wherein said subject has a level of A4 that is less than 1.75 times an upper limit of a reference range of A4 and a level of ACTH that is less than 1.75 times an upper limit of a reference range of ACTH.

10. The method of one of claims 1-3, wherein said subject has a level of 17-OHP that is less than 1.5 times an upper limit of a reference range of 17-OHP and a level of ACTH that is less than 1.5 times an upper limit of a reference range of ACTH.

11. The method of one of claims 1-3, wherein said subject has a level of 17-OHP that is less than 1.5 times an upper limit of a reference range of 17-OHP and a level of A4 that is less than 1.5 times an upper limit of a reference range of A4.

12. The method of one of claims 1-3, wherein said subject has a level of A4 that is less than 1.5 times an upper limit of a reference range of A4 and a level of ACTH that is less than 1.5 times an upper limit of a reference range of ACTH.

13. The method of one of claims 1-3, wherein said subject has a level of 17-OHP that is less than 1.25 times an upper limit of a reference range of 17-OHP and a level of ACTH that is less than 1.25 times an upper limit of a reference range of ACTH.

14. The method of one of claims 1-3, wherein said subject has a level of 17-OHP that is less than 1.25 times an upper limit of a reference range of 17-OHP and a level of A4 that is less than 1.25 times an upper limit of a reference range of A4.

15. The method of one of claims 1-3, wherein said subject has a level of A4 that is less than 1.25 times an upper limit of a reference range of A4 and a level of ACTH that is less than 1.25 times an upper limit of a reference range of ACTH.

16. The method of one of claims 1-3, wherein said subject has a level of 17-OHP that is less than one times an upper limit of a reference range of 17-OHP and a level of ACTH that is less than one times an upper limit of a reference range of ACTH.

17. The method of one of claims 1-3, wherein said subject has a level of 17-OHP that is less than one times an upper limit of a reference range of 17-OHP and a level of A4 that is less than one times an upper limit of a reference range of A4.

18. The method of one of claims 1-3, wherein said subject has a level of A4 that is less than one times an upper limit of a reference range of A4 and a level of ACTH that is less than one times an upper limit of a reference range of ACTH.

19. The method of any of claims 1-18, further comprising reducing said dose of said steroid or a pharmaceutically acceptable salt thereof in a 5 mg increment.

20. A method of treating Congenital Adrenal Hyperplasia (CAH), comprising administering a steroid or a pharmaceutically acceptable salt thereof and a corticotropin-releasing factor type-1 (CRF1) antagonist or a pharmaceutically acceptable salt thereof to a subject, wherein when said subject has a level of at least one steroid hormone that is more than an upper limit of a reference range of said at least one steroid hormone level, a dose of said steroid or a pharmaceutically acceptable salt thereof is not reduced compared to a dose of said steroid or a pharmaceutically acceptable salt thereof administered to a CAH patient that does not receive said CRF1 antagonist or a pharmaceutically acceptable salt thereof.

21. The method of claim 20, wherein said subject has a level of said at least one steroid hormone that is more than two times an upper limit of a reference range of said at least one steroid hormone level.

22. The method of claim 20, wherein said subject has a level of said at least one steroid hormone that is more than 1.75 times an upper limit of a reference range of said at least one steroid hormone level.

23. The method of claim 20, wherein said subject has a level of said at least one steroid hormone that is more than 1.5 times an upper limit of a reference range of said at least one steroid hormone level.

24. The method of claim 20, wherein said subject has a level of said at least one steroid hormone that is more than 1.25 times an upper limit of a reference range of said at least one steroid hormone level.

25. The method of claim 20, wherein said steroid hormone is selected from the group consisting of adrenocorticotropic hormone (ACTH), androstenedione (A4), 17-hydroxyprogesterone (17-OHP), deoxycorticosterone, 11-deoxycortisol, cortisol, corticosterone, aldosterone, pregnenolone, 17α-hydroxy pregnenolone, progesterone, dehydroepiandrosterone, androstenediol, testosterone, dihydrotestosterone, estrone, estradiol, and estriol.

26. The method of any of claims 20-25, further comprising maintaining said dose of said steroid or a pharmaceutically acceptable salt thereof until said levels of at least two steroid hormones are less than 2 times said upper limit of said reference ranges of said two steroid hormones respectively.

27. The method of any of claims 20-25, further comprising reducing said dose of said steroid or a pharmaceutically acceptable salt thereof in a 5 mg increment when said levels of at least two steroid hormones are less than 2 times said upper limit of said reference ranges of said two steroid hormones respectively.

28. The method of claim 27, wherein said at least two steroid hormones comprise 17-OHP and ACTH.

29. The method of claim 27, wherein said at least two steroid hormones comprise 17-OHP and A4.

30. The method of claim 27, wherein said at least two steroid hormones comprise A4 and ACTH.

31. The method of any of claims 1-30, wherein said reference range of ACTH or A4 level of said subject is controlled by a factor selected from the group consisting of age, sex, menopausal status, laboratory setting, time of a day and combinations thereof.

32. The method of any of claims 1-30, wherein said level of ACTH or A4 is 2 times, 1.5 times, 1.25 times, or 1 times said upper limit of said normal range of ACTH or A4 level of said subject.

33. The method of any of claims 1-30, wherein said amount of said steroid or a pharmaceutically acceptable salt thereof comprises a range of about 10 mg to about 80 mg of hydrocortisone equivalents, wherein said steroid or a pharmaceutically acceptable salt thereof is administered daily.

34. The method of any of claims 1-30, wherein said amount of said CRF1 antagonist or a pharmaceutically acceptable salt thereof comprises a range of about 10 mg to about 200 mg, wherein said CRF1 antagonist or a pharmaceutically acceptable salt thereof is administered daily.

35. The method of any of claims 1-34, wherein said subject is treated for a period of about 4 weeks to about 36 weeks.

36. The method of any of claims 1-34, wherein after said subject is treated for a period of about 4 weeks to about 36 weeks, said subject is reassessed to determine if a different method of treating CAH should be administered.

37. The method of any one of claims 1-36, wherein said CRF1 antagonist or pharmaceutically acceptable salt thereof is selected from the group consisting of: Antalarmin hydrochloride, Pfizer CP 154526, CP 376395 hydrochloride, NBI 27914 hydrochloride, NBI 35965 hydrochloride, NGD 98-2 hydrochloride, Pexacerfont, R 121919 hydrochloride, SNO03.

38. The method of any one of claims 1-37, wherein said CRF1 antagonist is a compound of Formula (I): 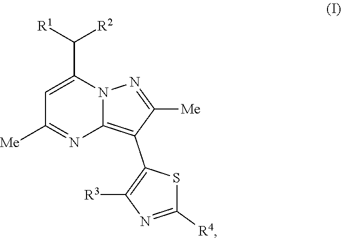
or a pharmaceutically acceptable salt thereof, wherein:
R¹ and R² are independently ethyl or n-propyl;
R³ is hydrogen, Cl, Br, methyl, trifluoromethyl, or methoxy; and
R⁴ is hydrogen, Br, R^{a}R^{b}N—, methoxymethyl, n-butyl, acetamido, pyridin-4-yl, morpholin-4-yl, 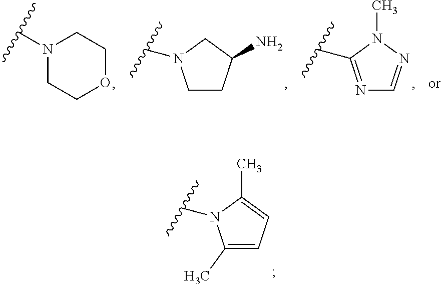
and
R^{a} and R^{b} are independently hydrogen, C₁-C₃alkyl, H₂NCH₂CH₂—, (CH₃)₃COC(O)NHCH₂CH₂—, or CH₃CH₂CH₂NHCH₂CH₂—.

39. The method of claim 38, wherein R³ is Cl, Br, methyl, or trifluoromethyl.

40. The method of claim 38 or 39, wherein R³ is Cl, Br, or methyl.

41. The method of any one of claims 38-40, wherein R⁴ is Br, R^{a}R^{b}N—, pyridin-4-yl, morpholin-4-yl, or

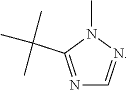

42. The method of any one of claims 38-40, wherein R⁴ is morpholin-4-yl or

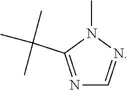

43. The method of any one of claims 38-40, wherein R⁴ is hydrogen, Br, R^{a}R^{b}N— and R^{a} and R^{b} are independently C₁-C₃alkyl.

44. The method of claim 38, wherein the compound is 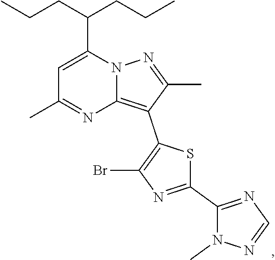 [SMILES: CCCC(CCC)c1cc(C)nc2c(-c3sc(-c4ncnn4C)nc3Br)c(C)nn12]
or a pharmaceutically acceptable salt thereof.

45. The method of claim 38, wherein the compound is 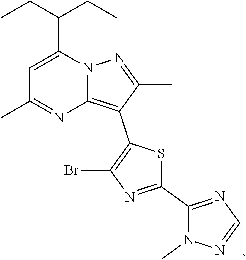 [SMILES: CCC(CC)c1cc(C)nc2c(-c3sc(-c4ncnn4C)nc3Br)c(C)nn12]
or a pharmaceutically acceptable salt thereof.

46. The method of claim 38, wherein the compound is 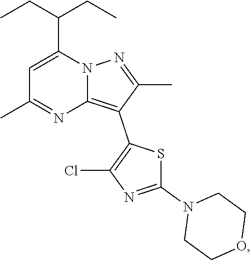 [SMILES: CCC(CC)c1cc(C)nc2c(-c3sc(N4CCOCC4)nc3Cl)c(C)nn12]
or a pharmaceutically acceptable salt thereof.

47. The method of any one of claims 1-46, wherein the CRF₁ antagonist or pharmaceutically acceptable salt is administered in a dose of about 50 mg to about 200 mg total daily dose to the subject.

48. The method of any one of claims 1-46, wherein the CRF₁ antagonist or pharmaceutically acceptable salt is administered in a dose of about 200 mg total daily dose to the subject.

49. The method of any one of claims 1-46, wherein the CRF₁ antagonist or pharmaceutically acceptable salt is administered in a dose of about 150 mg total daily dose to the subject.

50. The method of any one of claims 1-46, wherein the CRF₁ antagonist or pharmaceutically acceptable salt is administered in a dose of about 100 mg total daily dose to the subject.

51. The method of any one of claims 1-46, wherein the CRF1 antagonist or pharmaceutically acceptable salt is administered in a dose of about 75 mg total daily dose to the subject.

52. The method of any one of claims 1-46, wherein the CRF₁ antagonist or pharmaceutically acceptable salt is administered in a dose of about 50 mg total daily dose to the subject.

53. The method of any one of claims 1-46, wherein the CRF1 antagonist or pharmaceutically acceptable salt is administered in a dose of about 25 mg total daily dose to the subject.

54. The method of any one of claims 1-46, wherein the CRF₁ antagonist or pharmaceutically acceptable salt is in the form of microparticles.

55. The method of claim 54, wherein the average size of the microparticles is between about 1 μm to about 20 μm.

56. The method of claim 54, wherein the average size of the microparticles is between about 5 μm to about 15 μm.

57. The method of claim 54, wherein the average size of the microparticles is less than about 10 μm.

58. The method of any one of claims 1-56, wherein the CRF1 antagonist or pharmaceutically acceptable salt thereof is administered as a pharmaceutical composition.

59. The method of any one of claims 1-56, wherein the steroid or a pharmaceutically acceptable salt thereof is administered as a pharmaceutical composition.

60. The method of claim 58 or 59, wherein the pharmaceutical composition is in the form of a capsule or a tablet.

61. The method of claim 60, wherein the capsule is a hard gelatin capsule.

62. The method of claim 60, wherein the capsule is a soft gelatin capsule.

63. The method of any one of claims 60-62, wherein the capsule is formed using materials selected from the group consisting of natural gelatin, synthetic gelatin, pectin, casein, collagen, protein, modified starch, polyvinylpyrrolidone, acrylic polymers, cellulose derivatives, and any combinations thereof.

64. The method of any one of claims 58-63, wherein the pharmaceutical composition is free of additional excipients.

65. The method of any one of claims 58-63, wherein the pharmaceutical composition further comprises one or more pharmaceutically acceptable excipients.

66. The method of claim 60, wherein the pharmaceutical composition is in the form of a tablet.

67. The method of claim 66, wherein the pharmaceutical composition further comprises one or more pharmaceutically acceptable excipients.

68. The method of any one of claims 1-66, wherein the steroid or pharmaceutically acceptable salt thereof is a glucocorticoid or a pharmaceutically acceptable salt thereof.

69. The method of claim 68, wherein the glucocorticoid or a pharmaceutically acceptable salt thereof is prednisone, cortisone, prednisolone, triamcinolone, methylprednisolone, betamethasone, dexamethasone, hydrocortisone or a pharmaceutically acceptable salt thereof.

70. The method of any one of claims 1-69, wherein levels of steroid hormones in the subject are determined from a biological sample from the subject.

71. The method of claim 70, wherein the biological sample is selected from the group of blood, blood fractions, plasma, serum, and saliva.

72. The method of claim 70, wherein the biological sample is obtained non-invasively.

73. The method of any one of claims 1-72, wherein said subject is tested in the morning.

74. The method of any one of claims 1-72, wherein said subject is tested to determine morning levels of A4 and ACTH.

75. The method of any one of claims 1-72, wherein the subject is a pediatric patient.

76. The method of any one of claims 1-72, wherein the subject is from about 0 years old to about 18 years old.

77. The method of any one of claims 1-72, wherein the subject is an adult patient.

78. The method of any one of claims 1-77, wherein the steroid and the CRF₁ antagonist are administered concurrently.

79. The method of any one of claims 1-77, wherein the steroid and the CRF₁ antagonist are administered in one pharmaceutical composition.

80. The method of any one of claims 1-77, wherein the steroid and the CRF₁ antagonist are administered concurrently in separate pharmaceutical compositions.

81. The method of any one of claims 1-77, wherein the steroid and the CRF₁ antagonist are administered sequentially.

82. The method of claim 81, wherein the steroid and the CRF₁ antagonist are administered sequentially within 24 hours.

83. The method of claim 81, wherein the steroid and the CRF₁ antagonist are administered sequentially within 8 hours.

84. The method of claim 81, wherein the steroid and the CRF₁ antagonist are administered sequentially within 2 hours.

85. The method of claim 81, wherein the steroid and the CRF₁ antagonist are administered sequentially within 10 minutes.

86. The method of any one of claims 1-85, wherein CAH is classic CAH.

87. The method of any one of claims 1-85, wherein CAH is non-classic CAH.

88. A composition comprises a steroid or a pharmaceutically acceptable salt thereof and a corticotropin-releasing factor type-1 (CRF₁) antagonist or a pharmaceutically acceptable salt thereof, wherein said CRF₁ antagonist or pharmaceutically acceptable salt thereof is selected from the group consisting of: Antalarmin hydrochloride, Pfizer CP 154526, CP 376395 hydrochloride, NBI 27914 hydrochloride, NBI 35965 hydrochloride, NGD 98-2 hydrochloride, Pexacerfont, R 121919 hydrochloride, SN003.

89. The composition of claim 88, wherein said a steroid or a pharmaceutically acceptable salt thereof is exogenous glucocorticoids (GC) or a pharmaceutically acceptable salt thereof.

90. The composition of either claim 88 or 89, wherein said CRF₁ antagonist is a compound of Formula (I): 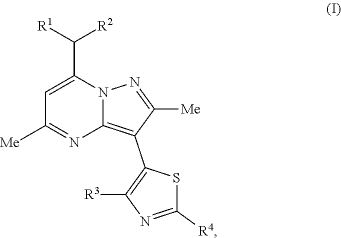
or a pharmaceutically acceptable salt thereof, wherein:
R¹ and R² are independently ethyl or n-propyl;
R³ is hydrogen, Cl, Br, methyl, trifluoromethyl, or methoxy; and
R⁴ is hydrogen, Br, R^{a}R^{b}N—, methoxymethyl, n-butyl, acetamido, pyridin-4-yl, morpholin-4-yl, 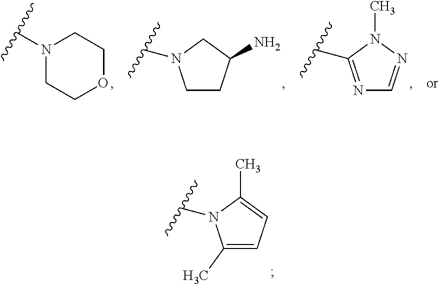
and
R^{a} and R^{b} are independently hydrogen, C₁-C₃alkyl, H₂NCH₂CH₂—, (CH₃)₃COC(O)NHCH₂CH₂—, or CH₃CH₂CH₂NHCH₂CH₂—.

91. The composition of claim 90, wherein R³ is Cl, Br, methyl, or trifluoromethyl.

92. The composition of claim 90 or 91, wherein R³ is Cl, Br, or methyl.

93. The composition of any one of claims 90-92, wherein R⁴ is Br, R^{a}R^{b}N—, pyridin-4-yl, morpholin-4-yl, or

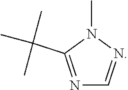

94. The composition of any one of claims 90-92, wherein R⁴ is morpholin-4-yl or

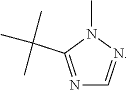

95. The composition of any one of claims 90-92, wherein R⁴ is hydrogen, Br, R^{a}R^{b}N— and R^{a} and R^{b} are independently C₁-C₃alkyl.

96. The composition of claim 90, wherein the compound is 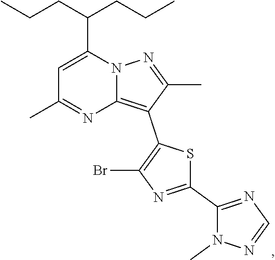 [SMILES: CCCC(CCC)c1cc(C)nc2c(-c3sc(-c4ncnn4C)nc3Br)c(C)nn12]
or a pharmaceutically acceptable salt thereof.

97. The composition of claim 90, wherein the compound is 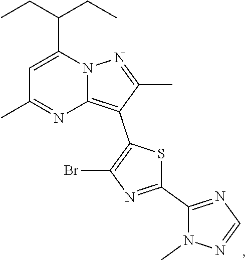 [SMILES: CCC(CC)c1cc(C)nc2c(-c3sc(-c4ncnn4C)nc3Br)c(C)nn12]
or a pharmaceutically acceptable salt thereof.

98. The composition of claim 90, wherein the compound is 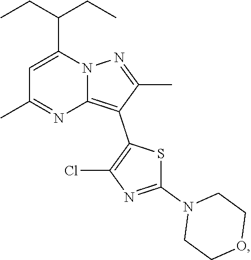 [SMILES: CCC(CC)c1cc(C)nc2c(-c3sc(N4CCOCC4)nc3Cl)c(C)nn12]
or a pharmaceutically acceptable salt thereof.

99. The composition of any one of claims 90-98, wherein the CRF₁ antagonist or pharmaceutically acceptable salt is administered in a dose of about 50 mg to about 200 mg total daily dose to the subject.

100. The composition of any one of claims 90-98, wherein the CRF₁ antagonist or pharmaceutically acceptable salt is administered in a dose of about 200 mg total daily dose to the subject.

101. The composition of any one of claims 90-98, wherein the CRF₁ antagonist or pharmaceutically acceptable salt is administered in a dose of about 150 mg total daily dose to the subject.

102. The composition of any one of claims 90-98, wherein the CRF₁ antagonist or pharmaceutically acceptable salt is administered in a dose of about 100 mg total daily dose to the subject.

103. The composition of any one of claims 90-98, wherein the CRF1 antagonist or pharmaceutically acceptable salt is administered in a dose of about 75 mg total daily dose to the subject.

104. The composition of any one of claims 90-98, wherein the CRF₁ antagonist or pharmaceutically acceptable salt is administered in a dose of about 50 mg total daily dose to the subject.

105. The composition of any one of claims 90-98, wherein the CRF1 antagonist or pharmaceutically acceptable salt is administered in a dose of about 25 mg total daily dose to the subject.
